(19) United States
(12) Patent Application Publication
(10) Pub. No.: US 2024/0066086 A1
(43) Pub. Date: Feb. 29, 2024

(54) Topical Hyperpigmentation Compositions and Method of Use

(71) Applicant: Allergan Sales, LLC, North Chicago, IL (US)

(72) Inventors: Tony Wang, Laguna Woods, CA (US); Kuniko Kadoya, San Diego, CA (US); Prithwiraj Maitra, Ladera Ranch, CA (US); Rahul Mehta, San Marcos, CA (US); Elizabeth Makino, Carlsbad, CA (US)

(73) Assignee: Allergan Sales, LLC, North Chicago, IL (US)

(21) Appl. No.: 18/458,248

(22) Filed: Aug. 30, 2023

Related U.S. Application Data
(60) Provisional application No. 63/373,891, filed on Aug. 30, 2022.

(51) Int. Cl. A61K 36/282; A61K 9/00; A61K 31/01; A61K 31/05; A61K 31/075; A61K 31/191; A61K 31/196; A61K 31/197; A61K 31/205; A61K 31/343; A61K 31/351; A61K 31/375; A61K 31/401; A61K 31/4015; A61K 31/455; A61K 31/4709; A61K 31/6615; A61K 31/728; A61K 31/765; A61K 36/03; A61K 36/42; A61K 36/48; A61K 36/73; A61K 36/87; A61K 36/886; A61K 38/07; A61K 45/06; A61P 17/00

(52) U.S. Cl. CPC A61K 36/282; A61K 9/0014; A61K 31/01; A61K 31/05; A61K 31/075; A61K 31/191; A61K 31/196; A61K 31/197; A61K 31/205; A61K 31/343; A61K 31/351; A61K 31/375; A61K 31/401; A61K 31/4015; A61K 31/455; A61K 31/4709; A61K 31/6615; A61K 31/728; A61K 31/765; A61K 36/03; A61K 36/42; A61K 36/48; A61K 36/73; A61K 36/87; A61K 36/886; A61K 38/07; A61K 45/06; A61P 17/00

(57) ABSTRACT

The present invention relates to topical compositions that can be used to treat melanin or pigmentation disorders and methods of using the compositions.

### CROSS-REFERENCE TO RELATED APPLICATIONS

This application claims the benefits of U.S. Provisional Application Ser. No. 63/373,891, filed on Aug. 30, 2022, the disclosure of which is incorporated herein by reference in its entirety.

### FIELD OF THE INVENTION

The present invention relates to topical compositions and methods for skincare treatment employing the topical compositions disclosed herein. More specifically, the topical compositions and methods for skincare disclosed herein are cosmetic compositions or formulations for treating pigmentation conditions of the skin such as melasma, dark spots, skin tone unevenness and brightness. The topical compositions may be applied to various areas of human skin, such as a submental region, face, neck, chest, arm, leg, hand or combination thereof, which exhibit the pigmentation condition and thereby treat the pigmentation condition.

### BACKGROUND

Melanin in humans is the primary determinant of skin color. Melanin in skin is produced by melanocytes in the epidermis in response to triggers, such as increased sun exposure or other physical or chemical exposure, induced inflammation or endocrine changes associated with pregnancy, lactation, physiological stress, medication, etc. Hyperpigmentation can be triggered by a variety of other stimuli such as skin infections, allergic reactions, mechanical injuries, reactions to medications, phototoxic eruptions, trauma (e.g., burns), inflammatory diseases (e.g., lichen planus, lupus erythematosus and atopic dermatitis), as well as reactions to energy emitting devices, including electromagnetic devices such as ultrasound, radiofrequency, lasers, light-emitting diodes and visible light therapy, as well as microdermabrasion reactions.

If the number of melanocytes in the epidermis is increased or if the melanocytes are over stimulated, an increase in melanin can occur leading to hyperpigmentation of the skin. Often, hyperpigmentation is an unwanted condition having adverse impact on patient's psychological well-being. Topical products for treating, reducing or elimination of unwanted hyperpigmentation of the skin are described in the art such as U.S. Pat. Nos. 8,236,288; 7,429,391; 8,968,755; 10,806,693; and 11,154,493.

There is a need for improved topical compositions that can treat pigmentation conditions.

### SUMMARY OF THE INVENTION

The present invention relates to topical compositions that can be used to treat skin pigmentation concerns, especially hyperpigmentation conditions such as, but not limited to, melasma and dark spots on human skin.

The topical compositions may be applied to various areas of human skin, such as a submental region, face, neck, chest, arm, leg, hand or combination thereof, and specifically directly to the hyperpigmentation area.

The topical compositions comprise: (i) one or more bisbenzylisoquinoline alkaloid such as neferine, liensinine, isoliensine, nelumboferine, nelumborine, N-norisoliensinine, 6-hydroxynorisoliensinine and combinations thereof, (ii) at least one additional agent that inhibits the production of melanin; and (iii) one or more conventional topical/cosmetic carriers, auxiliaries or excipients such as one or more thickeners/viscosity enhancing agents, film forming polymers, emollients, chelating agents, humectants, preservatives, pH adjusting agents, buffering agents, solvents, surfactants, emulsifiers, sunscreen agents and combinations of the foregoing.

The bisbenzylisoquinoline alkaloid may be prepared synthetically or obtained from a natural source such from the Nelumbo nucifera (Sacred lotus) plant or another rich source of these family of compounds, or a combination of thereof; or an extract prepared from a natural source of such compounds, such as parts of Nelumbo nucifera plant. In some embodiments, the one or more bisbenzylisoquinoline alkaloids are obtained from the embryo, flower, seed, rhizome, or combination thereof of the Nelumbo nucifera plant. In some embodiments the topical compositions will comprise at least about 0.0005 wt %, preferably at least about 0.00075 wt % and more preferably at least about 0.001 wt % of one or more bisbenzylisoquinoline alkaloids.

The topical compositions may be a solution, suspension, dispersion, emulsion, gel, cream, lotion, ointment or serum.

The topical compositions may be applied to various skin areas of a human subject, such as a submental region, face, neck, chest, arm, leg, hand or combination thereof, and specifically directly to the hyperpigmentation area at least once, twice, thrice or more times a day for a period of at least 1 to 30 days or longer.

The present invention further relates to methods of using the topical compositions to treat skin pigmentation concerns, especially hyperpigmentation conditions such as, but not limited to, melasma and dark spots on human skin.

### BRIEF DESCRIPTION OF THE DRAWINGS

FIG. 1 shows the mean percent change from baseline in overall hyperpigmentation for the study described in Example 6.

FIG. 2 shows the mean percent change from baseline in skin tone evenness for the study described in Example 6.

FIGS. 3A-3D show the mean percent change from baseline in overall hyperpigmentation for the ethnic subgroups described in Example 6.

FIG. 4 shows the mean scores and mean improvement for mMASI in the melasma subgroup for the study described in Example 6.

FIG. 5 shows the mean scores for MelasQol in the melasma subgroup for the study described in Example 6.

FIG. 6 shows the mean percent change from baseline in overall hyperpigmentation for the study described in Example 7.

FIG. 7 shows the mean percent change from baseline in skin tone evenness for the study described in Example 7.

FIG. 8 shows the mean L* scores of dark spot brightness for the study described in Example 7.

FIG. 9A shows the mean percent change from baseline in overall dark spot intensity, dark spot contrast and dark spot size for the study described in Example 7.

FIG. 9B shows the mean percent change from baseline in overall dark spot intensity, dark spot contrast and dark spot size for the PIH subgroup of the study described in Example 7.

FIG. 9C shows the mean percent change from baseline in overall dark spot intensity, dark spot contrast and dark spot size for the solar lentigines subgroup of the study described in Example 7.

FIG. 10 reports a summary of the participant self-assessment questionnaire at week 12 for the study described in Example 7.

FIG. 11 reports tolerability values for the study described in Example 7.

FIG. 12 shows the mean percent change from baseline in overall hyperpigmentation, tactile roughness and MASI for the study described in Example 8.

FIG. 13 shows the mean percent improvement from baseline in radiance assessment for the study described in Example 8.

FIG. 14 shows the moisturization/hydration scores before and after the first treatment procedure described in Example 8.

### DETAILED DESCRIPTION OF THE INVENTION

Before the present invention is further described, it is to be understood that this invention is not limited to the particular embodiments described. It is also to be understood that the terminology used herein is for the purpose of describing particular embodiments only, and is not intended to be limiting.

It should be noted that as used herein, the singular forms “a,” “an,” and “the” include plural referents unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are encompassed within the invention, subject to any specifically excluded limit in the stated range. Where the stated range includes one or both of the limits, ranges excluding either or both of those included limits are also included in the invention.

Examples of the bisbenzylisoquinoline alkaloid that may be used in the present invention include, but are not limited to neferine, liensinine, isoliensine, nelumboferine, nelumborine, N-norisoliensinine, 6-hydroxynorisoliensinine and combinations thereof. The chemical structures and molecular formulas for neferine, liensinine, isoliensine, nelumboferine, nelumborine, N-norisoliensinine, 6-hydroxynorisoliensinine can be found in Menendez-Perdoino, Ivette M., and Peter J. Facchini. “Benzylisoquinoline alkaloids biosynthesis in sacred lotus.” Molecules 23.11 (2018): 2899, which is incorporated herein by reference. The bisbenzylisoquinoline alkaloid may be prepared synthetically or obtained from a natural source such from the Nelumbo nucifera (Sacred lotus), or a combination of thereof. In some embodiments the one or more bisbenzylisoquinoline alkaloids are obtained from the embryo, flower, seed, rhizome, or combination thereof of the Nelumbo nucifera plant and preferably the seed. The one or more bisbenzylisoquinoline alkaloids in the present invention may also be present in the topical composition as part of a plant extract, for example as part of an extract obtained from the embryo, flower, seed, rhizome, or combination thereof of the Nelumbo nucifera plant and preferably the seed.

The topical compositions of the present invention may comprise at about 0.0005 wt % to about 1.0 wt %, preferably about 0.00075 wt % to about 0.5 wt % and more about 0.001 wt % to about 0.1 wt % of one or more bisbenzylisoquinoline alkaloids. In certain embodiments, the topical compositions comprise at least about 0.0005 wt %, preferably at least about 0.00075 wt % and more preferably at least about 0.001 wt % of neferine. In certain embodiments, the topical compositions comprise at least about 0.0005 wt %, preferably at least about 0.00075 wt % and more preferably at least about 0.001 wt % of liensinine. In certain embodiments, the topical compositions comprise at least about 0.0005 wt %, preferably at least about 0.00075 wt % and more preferably at least about 0.001 wt % of isoliensine. In certain embodiments, the topical compositions comprise a combination of neferine and liensinine, preferably in the form of a botanical extract, such as a lotus seed extract also known as Nelumbo nucifera germ or seed extract.

In certain embodiments, the topical compositions of the present invention comprise both neferine and liensinine wherein the neferine comprise about 0.0005 wt % to about 1.0 wt %, preferably about 0.00075 wt % to about 0.5 wt % and more about 0.001 wt % to about 0.1 wt % of the topical composition and the liensinine comprises about 0.0005 wt % to about 1.0 wt %, preferably about 0.00075 wt % to about 0.5 wt % and more about 0.001 wt % to about 0.1 wt % of the topical composition.

Neferine and liensinine were tested in vitro and were shown to significantly inhibit melanogenesis in human melanoma cell line MNT-1 cells, primary human melanocyes and 3-D dark pigmented epidermis model Melanoderm™ MEL0300B tissue and therefore should inhibit and/or reduce production of melanin in human skin cells in vivo. In the MNT-1 cell in vitro tests, neferine and liensinine reduced melanin production after seven days by at least 50%, 60%, 70%, 80% or greater at concentrations ranging from 0.1 μM to 5 μM.

The topical compositions of the present invention further comprise at least one additional agent that inhibits the production of melanin. These agents may inhibit the activation or proliferation of melanocytes, inhibit the biosynthesis of melanin, inhibit transport of melanin from melanocytes to epidermal keratinocytes and/or remove or reduce the melanin present in the epidermis. Examples of agents that can be used to inhibit the product of melanin include, but are not limited to, tranexamic acid, phenylethyl resorcinol, peptides such as di-, tri-, tetra and hexa-peptides (i.e., such as tetrapeptide-30), kojic diplamitate (aka hexadecanoic acid), vitamin C and derivatives thereof such as tetrahexydecyl ascorbate, plankton extracts, sclareolide and other skin whitening compounds or botanical extracts such as artemisia capillaris flower extract, pyrus malus (apple) fruit extract and those described in U.S. Pat. Nos. 8,236,288 and 7,429,391 which are incorporated herein by reference.

In certain embodiments at least one additional agent that inhibits the production of melanin comprises tranexamic acid and at least one or more additional agents, i.e. at least a third, fourth etc. agent that inhibits the production of melanin. The tranexamic acid may be present in the topical compositions in an amount ranging from about 0.05 wt % to about 15 wt %, preferably about 0.1 wt % to about 12.5 wt % and more about 1 wt % to about 10 wt % of the topical composition. The at least one or more further agents, i.e. at least a third, fourth, fifth etc. agent, that inhibits the production of melanin may be present in the topical compositions in an amount ranging from about 0.05 wt % to about 15 wt %, preferably about 0.1 wt % to about 12.5 wt % and more about 1 wt % to about 10 wt % of the topical composition. Examples of the least one or more additional agents, i.e. at least a third, fourth, fifth etc. agent, that inhibits the production of melanin include but are not limited to resorcinol, phenylethyl resorcinol, tri- and tetra-peptides (i.e., such as tetrapeptide-30), kojic diplamitate (aka hexadecanoic acid), vitamin C and derivatives thereof such as tetrahexydecyl ascorbate, plankton extracts, sclareolide and combinations thereof.

In some embodiments of the present invention, the topical compositions may further comprise one or more antioxidants, one or more anti-inflammatory agents, one or more exfoliants, one or more skin conditioning agents and combinations thereof.

As used herein the term antioxidant broadly refers to a compound that retards oxidation or degradation. The antioxidant may retard the oxidation or degradation of one or more ingredients in the topical composition and/or may retard the oxidation or degradation of the components of the skin cells in the target area or application site. Unless specifically stated, the antioxidant may be a commonly known antioxidant such as those described in the United States Pharmacopeia or Handbook of Pharmaceutical Excipients, a botanical extract or a combination thereof. The topical compositions in accordance with the present invention may comprise one or more antioxidants. In certain embodiments, the topical composition will comprise about 0.0005 wt % to about 10.00 wt %, preferably about 0.0007 wt % to about 7.5 wt % and more preferably about 0.001 wt % to about 5.00 wt % based on the total weight of the topical composition of one or more antioxidants. Examples of antioxidants that can be used with the topical compositions of the present invention include, but are not limited to, niacinamide, peptides, such as di-, tri-, tetra and hexa-peptides, acetyl cysteine, ascorbic acid polypeptide, ascorbyl dipalmitate, ascorbyl methylsilanol pectinate, ascorbyl palmitate, ascorbyl stearate, butylated hydroxyanisole (BHA), butylated hydroxytoluene (BHT), t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, Helianthus annus (sunflower) seed oil, hydroquinone, isooctyl thioglycolate, kojic acid, Laminaria digitata extract, magnesium ascorbate, magnesium ascorbyl phosphate, methylsilanol ascorbate, natural botanical anti-oxidants such as camellia sinnensis green tea extract or vitris vinifera (grape) callous culture extract, nordihydroguaiaretic acid, octyl gallate, phenylthioglycolic acid, potassium ascorbyl tocopheryl phosphate, potassium sulfite, propyl gallate, quinones, rosmarinic acid, Rosa mochata seed oil, Rosmarinus officinalis (rosemary) leaf extract, sodium ascorbyl phosphate, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, tremella fuciformis (mushroom) extract, tris(nonylphenyl)phosphite, and Zea mays (corn) oil. In certain embodiments, the topical compositions may comprise one or more antioxidants selected from the group consisting of niacinamide, Vitis vinifera flower cell extract, tocopherol acetate, acetyl tetrapeptide-2, lutein (aka xanthophyll), propyl gallate, hydroxyacetaphenone, sodium bisulfite, diethylhexyl syringylidenemalonate, BHA, BHT, Citrullus lanatus (Watermelon) fruit extract, Chamomilla recutita (matricaria) flower extract or combinations thereof.

In certain embodiments, the topical composition of the present invention may comprise one or more anti-inflammatory agents. In certain embodiments, the topical composition will comprise about 0.0005 wt % to about 5.00 wt %, preferably about 0.0007 wt % to about 2.5 wt % and more preferably about 0.001 wt % to about 1.00 wt % based on the total weight of the topical composition of one or more anti-inflammatory agents. Examples of anti-inflammatory agents properties include but are not limited to dipotassium glycyrrhizate, bisabolol, allantoin, aloe extracts, panthenol, Chamomilla recutita (matricaria) flower extract and materials derived from the following: phellodendron Amurense corte extract (PCE), Tanacetum parthenium commercially available under the tradename FEVERFEW, Zingiber officinate (ginger) root extract, ginko (Ginkgo biloba), centella asiatica extract, commercially available under the tradename MADECASSOSIDE, cotinus (cotinus coggygria), butterbur extract (Petasites hybridus), goji berry (Lycium barbarum), milk thistle extract (Silybum marianum), honeysuckle (Lonicera japonica), basalm of peru (Myroxylon pereirae), sage (Salvia officinalis), cranberry extract (Vaccinium oxycoccos), amaranth oil (Amaranthus cruentus), pomegranate (Punica granatum), yerbe mate (Ilex paraguariensis leaf extract), white lily flower extract (Lilium candidum), olive leaf extract (Olea europaea), phloretin (apple extract), oat flour (Avena sativa), hops (Humulus lupulus) extract, commercially available under the tradename LIFENOL, bugrane p (Ononis spinosa), licochalcone (licorice: glycyrrhiza inflate extract ingredient), and ginger extract, commercially available under the tradename SYMRELIEF, and combinations thereof. Additional examples may be found in U.S. Patent Application Publication Nos. 2020/0069562 and 2017/0128357, which are incorporated herein by reference. In certain embodiments, the topical compositions may comprise one or more anti-inflammatory agents selected from the group consisting of dipotassium glycyrrhizate, bisabolol, allantoin, aloe leaf extracts, panthenol, Chamomilla recutita (matricaria) flower extract, Avena sativa bran extract, or combinations thereof.

In certain embodiments, the topical composition of the present invention may comprise one or more exfoliants. In certain embodiments, the topical composition will comprise about 0.0005 wt % to about 5.00 wt %, preferably about 0.0007 wt % to about 2.5 wt % and more preferably about 0.001 wt % to about 1.00 wt % based on the total weight of the topical composition of one or more exfoliants. Examples of exfoliants include but are not limited to retinoids, alpha-hydroxy acids such as lactic acid, glycolic acid, malic acid, tartaric acid, citric acid, or any combination of any of the foregoing, and beta-hydroxy acids such as salicylic acid, polyhydroxy acids such lactobionic acid and gluconic acid. Examples of the exfoliants can also be found in U.S. Pat. Nos. 5,756,107; 9,161,958 and 9,782,334, which are incorporated herein by reference.

In certain embodiments, the topical composition of the present invention may comprise one or more skin conditioning agents. As used herein skin conditioning agents include compounds that hydrate or improve the texture and feel of skin and include humectants and moisturizers. In certain embodiments, the topical composition will comprise about 0.0005 wt % to about 15 wt %, preferably about 0.0007 wt % to about 10 wt % and more preferably about 0.001 wt % to about 7.5 wt % based on the total weight of the topical composition of one or more skin conditioning agents. Examples of skin conditioning agents include but are not limited to, lactic acid and other hydroxy acids and their salts, glycerin, propylene glycol, butylene glycol, sodium pyrrolidine carboxylic acid (“PCA”), Carbowax 200, Carbowax 400, and Carbowax 800, PPG-15 stearyl ether, lanolin alcohol, lanolin, lanolin derivatives, cholesterol, petrolatum, isostearyl neopentanoate, octyl stearate, mineral oil, isocetyl stearate, Ceraphyl 424 (myristyl myristate), octyl dodecanol, dimethicone (Dow Corning 200-100 cps), phenyl trimethicone (Dow Corning 556), Dow Corning 1401 (cyclomethicone and dimethiconol), and cyclomethicone (Dow Corning 344), and Miglyol 840 (manufactured by Huls; propylene glycol dicaprylate/dicaprate) guanidine, urea, glycolic acid, glycolate salts (e.g. ammonium and quaternary alkyl ammonium), salicylic acid, lactic acid, lactate salts (e.g., ammonium and quaternary alkyl ammonium), aloe vera in any of its variety of forms (e.g., aloe vera gel), polyhydroxy alcohols such as sorbitol, mannitol, xylitol, erythritol, glycerol, hexanetriol, butanetriol, propylene glycol, butylene glycol, hexylene glycol and the like, polyethylene glycols, sugars (e.g., melibiose), starches, sugar and starch derivatives (e.g., alkoxylated glucose, fructose, glucosamine), hyaluronic acid, lactamide monoethanolamine, acetamide monoethanolamine, panthenol. Additional examples of skin conditioning agents can be found in U.S. Pat. Nos. 5,804,203 and 8,741,357, which are incorporated herein by reference. In certain embodiments, the topical compositions may comprise one or more skin conditioning agents selected from the group consisting of glycerin, coconut alkanes, Butyrospermum parkii (shea) butter, ethylhexyl olivate, polyisobutene, squalene, cetearyl alcohol, arginine, trehalose, coco-caprylate/caprate, ethylhexylglycerin, sucrose, caprylic/capric triglyceride, sodium hyaluronate, hyaluronic acid, sorbitan isostearate, cyclopentasiloxane, PEG-10 dimethicone, dimethicone/vinyl dimethicone crosspolymer, hydrated silica, ceramide, silica, Dunaliella salina extract, caprylyl glycol, 1-2-hexanediol, aloe barbadensis leaf extract, butylene glycol, panthenol, lens esculenta (lentil) fruit extract, sodium PCA, Laminaria digitata extract, Artemisia vulgaris extract, isopentyldiol, betaine, Hamamelis virginiana (witch hazel) water, Avena sativa (oat) bran extract or combinations thereof.

Topical compositions described herein may also include one or more conventional topical/cosmetic carriers, auxiliaries and/or excipients such as solvents, preservatives, viscosity increasing agents, pH adjusting agents, aesthetic agents (i.e., compounds that improve the smell and/or appearance of the topical composition), surfactants, chelating agents, permeation enhancers, or combination thereof. The CTFA International Cosmetic Ingredient Dictionary and Handbook (2008), 12^{th} Edition, describes a wide variety of non-limiting cosmetic ingredients that may be used in embodiments of the present invention.

Examples of solvents that may be used in the topical compositions of the present invention include water and/or organic based solvents such as C₁-C₁₀ mono-alcohols (e.g., methanol, ethanol, isopropanol, benzyl alcohol, phenoxyethanol etc.) C₂-C₁₂ polyalcohols (e.g., ethylene glycol, propylene glycol, hexylene glycol, glycerin, etc.) or combinations thereof. In certain embodiments, the topical compositions of the present invention comprise about 20 wt % to about 90 wt %, preferably about 30 wt % to about 85 wt % and more preferably about 35 wt % to about 80 wt % of one or more solvents based on the total weight of the topical composition.

Examples of preservatives that may be included in the topical compositions of the present invention include, but are not limited to, potassium sorbate, acids, alcohols, glycols, parabens, quaternary-nitrogen containing compounds, isothiazolinones, aldehyde-releasing compounds and halogenated compounds. Illustrative alcohols include phenoxyethanol, isopropyl alcohol, and benzyl alcohol; illustrative glycols include propylene, butylene and pentylene glycols; illustrative parabens include (also known as parahydroxybenzoic acids) methyl, propyl and butyl parabens; illustrative quaternary nitrogen containing compounds include benzalkonium chloride, Quartenium 15; illustrative isothiazoles include methylisothiazoline, methychlorolisothiazoline; illustrative aldehyde releasing agents include DMDM hydantion, imiadolidinyl urea and diazolidinyl urea; illustrative antioxidants include butylated hydroxytoluene, tocopherol and illustrative halogenated compounds include triclosan and chlorohexidene digluconate.

Examples of viscosity enhancing agents that may be used in the topical compositions of the present invention include but are not limited to carboxylic acid polymers such as carbomers, which are homopolymers of acrylic acid crosslinked with allyl ethers of sucrose or pentaerytritol, available as under the tradename CARBOPOL®. In addition, other suitable carboxylic acid polymeric agents include ULTREZ® 10 and copolymers of C₁₀-C₃₀ alkyl acrylates with one or more monomers of acrylic acid, methacrylic acid, or one of their short chain (i.e., C₁-C₄) esters, wherein the crosslinking agent is an allyl ether of sucrose or pentaerytritol. These copolymers are known as acrylates/C₁₀-C₃₀ alkyl acrylate crosspolymers and are commercially available as CARBOPOL® 1342, CARBOPOL® 1382, PEMULEN TR-1, and PEMULEN TR-2.

Additional viscosity enhancing agents that may also be used in the topical compositions of the present invention include crosslinked polyacrylate polymers, including both cationic and nonionic polymers, with the cationic being generally preferred. Examples of useful crosslinked nonionic polyacrylate polymers and crosslinked cationic polyacrylate polymers are those described in U.S. Pat. Nos. 5,100,660, 4,849,484, 4,835,206, 4,628,078, 4,599,379 and EP228868.

Further examples of viscosity enhancing agents that may also be used in the topical compositions of the present invention include a wide variety of polysaccharides. “Polysaccharides” refer to gelling agents that contain a backbone of repeating sugar (i.e., carbohydrate) units. Non-limiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. Other useful polysaccharides include scleroglucans comprising a linear chain of (1-3) linked glucose units with a (1-6) linked glucose every three units, a commercially available example of which is CLEARGEL™ CS11 from Michel Mercier Products Inc.

Still further examples of viscosity enhancing agents that may also be used in the topical compositions of the present invention include thickening and/or gelling agents which are primarily derived from natural sources. Non-limiting examples of these naturally derived thickening and/or gelling agents include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

Additional examples of viscosity enhancing agents include silicone containing compounds, e.g., silicone oils, silicone elastomers and/or polyorganosiloxanes. Examples of the foregoing can be found in U.S. Pat. No. 10,286,030, which is incorporated herein by reference. In certain embodiments of the present invention the topical composition will comprise a silicone elastomer and/or polyorganosiloxanes, such as a dimethicone crosspolymer commercially available under the tradename DOWSIL™ EL-8040 ID Silicone Organic Blend, which will provide multiple properties including but not limited to increasing the viscosity and feel of the composition and provide skin conditioning benefits.

Examples of pH modifying agents that may be used in the topical compositions of the present invention include but are not limited to compounds that are added to raise or low the pH of the final topical composition as well as compounds that are added to buffer the final topical composition. Compounds that may be added to raise or lower the pH of the topical composition include but are not limited to phosphoric acid and/or phosphate salts, citric acid and/or citrate salts, hydroxide salts (i.e., calcium hydroxide, sodium hydroxide, potassium hydroxide) and amines, such as triethanolamine. Examples of compounds that buffer the topical compositions include, acetic acid and salts, citric acid and salts, boric acid and salts, and phosphoric acid and salts such as mono- and di-potassium phosphate, citric acid/sodium citrate, and dibasic sodium phosphate/citric acid.

Examples of aesthetic agents that may be used in the topical compositions of the present invention include but are not limited to fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11).

Examples of chelating agents that may be used in the topical compositions of the present invention include but are not limited to citric acid and salts thereof, disodium EDTA, tetrasodium EDTA, and phytic acid.

Additional specific examples of the carriers, auxiliaries and/or excipients of the foregoing classes can be found in U.S. Pat. Nos. 9,408,881; 10,286,030; and 10,493,011 and U.S. Patent Application Publication Nos. 2011/0158922; 2012/0076842; and 2015/0202139, all of which are incorporated herein by reference. The skilled artisan is aware that some of the carries, auxiliaries and/or excipients may be included in more than one of the foregoing classifications. Stated another way, some of the carriers, auxiliaries and/or excipients may impart one or more properties to the topical composition.

Topical compositions of the present invention may be a lotion, emulsion, cream, gel, ointment, foam, liquid, paste or other topically administrable form. In certain embodiments, a topical composition is a serum, gel, or liquid. Embodiments of the topical compositions may exhibit a viscosity of less than 100,000 cps, preferably less than 75,000 cps and more preferably less than 50,000 cps at 25° C. when tested using a conventional viscosity apparatus, such as a Brookfield RVT viscometer with spindle 2 and 20 rpms. In certain embodiments, the topical composition will exhibit a viscosity at 25° C. between about 3,000 cps and 75,000 cps, preferably about 5,000 cps and about 60,000 cps and more preferably about 7,000 cps and about 50,000 cps. In certain embodiments, the topical composition will exhibit a low viscosity at 25° C. between about 1 cps and 200 cps, preferably about 1 cps and about 100 cps and more preferably about 1 cps and about 50 cps.

The topical compositions of the present invention may be free of hydroquinone and steroids, especially corticosteroids. In certain aspects, the topical compositions of the present invention may contain a retinoid such as retinol, tretinoin, isotretinoin, alitretinoin, etretinate, acitretin, adapalene, bexarotene and tazarotene or the topical compositions may be free of retinoids, particularly free of tretinoin, isotretinoin, alitretinoin, etretinate, acitretin, adapalene, bexarotene and tazarotene.

Topical compositions of the present invention may be prepared by any method commonly known in the industry and may include blending, mixing, emulsifying, heating, cooling steps or any combination thereof. Examples of manufacturing methods can be found in U.S. Pat. No. 10,493,011 and U.S. Patent Application Publication No. 2011/0158922, which are incorporated herein by reference.

Topical compositions of the present invention may be packaged and dispensed in any suitable container. Containers can include a bottle, a tube (metal, plastic or laminate), a pressurized container, or pouches. The containers may include amounts of the topical composition for single or multiple applications. Containers may include indicia on its surface that instruct the subject on its application and use. Instructions for use may also be printed separately and packaged with the container comprising the topical composition.

Containers comprising multiple applications of the topical composition can dispense a pre-determined amount of the topical composition via a metered or calibrated spray, pump, or squeeze mechanism. In other embodiments, the container can be squeezed (e.g., metal, laminate, or plastic tube) to dispense a desired amount of the composition.

Lower viscosity topical compositions, i.e., topical compositions with viscosity of less than 200 cps, may be applied to the skin using a pad or wipe. A pad or wipe may be made of cotton, woven or non-woven polymeric material such as polyester, polypropylene, nylon, rayon or combinations thereof. The low viscosity topical composition can be applied to a pad or wipe and one or more pads or wipes may be packaged in a suitable container such as ajar, plastic bottle, or flexible packaging such as a single use foil package.

The topical compositions of the present invention are used for the treatment of melanin or pigmentation disorders, including but not limited to, treatment of hyperpigmentation or hypermelanosis disorders that affect melanin or pigmentation levels in the skin, treatment of skin tone uneveness, treatment of skin brightness and other skin pigmentation disorders known to those skilled in the art. Such disorders may be due to environmental stressors, such as excessive sun exposure, or physiological stressors, such as hormonal imbalance. Such disorders include melasma, chloasma, post-inflammatory hyperpigmentation, acanthosis nigricans, pigmented purpura, urticaria, pityriasis, solar lentigines, vitiligo, birthmarks, port-wine stains, dark spots, age spots, freckles or ephelides. In addition, the disclosed compositions may be used in conjunction with and/or for the treatment of skin disorders or trauma as a result of a mechanical injury or therapy, including but not limited to laser treatment, chemical peels, intense pulsed light, dermabrasion or cryotherapy.

In certain embodiments, the subject will apply the desired amount of the topical composition to a clean and preferably dry skin surface with the melanin or pigmentation disorder at least once a day, preferably twice or thrice a day. In certain embodiments, the subject will apply about 0.1 to about 2.0 grams, preferably 0.20 grams to about 1.5 grams and more preferably about 0.3 grams to about 1.6 grams. The amount will be adjusted depending upon surface area to be treated. In some embodiments, the topical composition in accordance with the present invention may be applied to the target area once, twice or thrice daily for at least 1 day, 2 days, 3 days, 4 days, 5 days, 6 days, 1 week, 2 weeks, 3 weeks, 1 month, 2 months, 3 months, 4 months, 5 months, 6 months, or more.

The topical compositions of the present invention may decrease melanin and/or melanocyte distribution and/or levels. For example, in the treatment of hyperpigmentation or hypermelanosis disorders, the disclosed compositions may decrease melanin and/or melanocyte distribution and/or levels by about 1%, about 2%, about 3%, about 4%, about 5%, about 6%, by about 7%, about 8%, about 9%, by about 10%, by about 12%, by about 15%, by about 20%, by about 25%, by about 30%, by about 35%, by about 35%, by about 40%, by about 45%, by about 50%, by about 55%, by about 60%, by about 65%, by about 70%, by about 75%, by about 80%, by about 85%, by about 90%, by about 95% or by about 100%. In another embodiment, melanin and/or melanocyte distribution may be decreased by about 5% to about 75%, preferably by about 10% to about 65% and more preferably by about 20% to about 50% after 1 to 4 weeks of treatment.

One or more of the topical compositions described herein may be used to treat various melanin or pigmentation disorders mentioned above such as melasma, post-inflammatory hyperpigmentation, solar lentigines, etc. For example, a composition as described herein may be used during a melanin or pigmentation disorder procedure conducted in a dermatologist or skin-care professional office (i.e., an in-office procedure). The in-office procedure include but are not limited to a chemical peel, laser/light therapy, micro needling and/or microdermabrasion. After the in-office procedure, the patient/subject will use the same or different topical composition as described herein to further treat the melanin or pigmentation disorders while at home or out of the office. For example, a first topical composition as described herein is used immediately before, during, or immediately after a microdermabrasion procedure. In this context, immediately before or immediately after means within 30, 25, 20, 15, 10, 5 minutes of the in-office procedure. Once the in-office procedure is completed, the patient/subject will apply one or more of the topical compositions described herein to the hyperpigmentation or treatment area, once, twice, thrice or more times a day while at home or out of the office for a period of time such as 1, 2, 3, 4, 5, 6, 7, 8, 9, 10, 11, 12 or more weeks. If the melanin or pigmentation disorder remains after the predetermined period of at home or out of office treatment, the described in-office and out of office treatment process may be repeated as necessary until the melanin or pigmentation disorder is reduced or eliminated.

The methods of treating the various melanin or pigmentation disorders with the topical compositions of the present invention as described herein may also include the administration of compositions comprising hydroquinone, retinoids and/or steroids, especially corticosteroids. For example, the topical compositions of the present invention may be used with a second topical composition comprising hydroquinone, retinoids and/or steroids. An example of a second topical composition, not in accordance with the present invention, that could be used is the U.S. Food and Drug Administration approved product TRI-LUMA® topical cream containing 0.01% fluocinolone acetonide, 4% hydroquinone and 0.05% tretinoin. In one aspect of the co-administration method, the topical compositions of the present invention may be applied to the patient's skin in need of treatment and after a period of time a second topical compositions comprising hydroquinone, retinoids and/or steroids can be applied to the same area on the patient's skin in need of treatment. In some embodiments of this co-administration aspect, the topical composition of the present invention can be applied to the patient's skin in need of treatment once or twice daily such as in the morning or evening and the second topical composition comprising hydroquinone, retinoids and/or steroids can be applied once or twice daily, preferably, 4, 6, 8, 10 or 12 hours after the application of the topical composition of the present invention.

The methods of treating the various melanin or pigmentation disorders with the topical compositions of the present invention as described herein may exclude or be conducted without the administration of compositions comprising hydroquinone, retinoids and/or steroids, especially corticosteroids. In certain aspects of this method of treating various melanin or pigmentation disorders, the method comprises the administration of the of topical compositions of the present invention for a period of time such as once, twice or thrice daily for 1, 2, 3, 4, 5, 6, 7, 8, 9, 10, 11, 12 weeks or longer and during the treatment regimen, the patient is: (i) not administered hydroquinone; (ii) not administered a retinoid such as tretinoin, isotretinoin, alitretinoin, etretinate, acitretin, adapalene, bexarotene and tazarotene; (iii) not administered a steroid; or (iv) any combination of (i), (ii) and (iii).

The methods of treating the various melanin or pigmentation disorders with the topical compositions of the present invention as described herein may further include the steps of: (i) cleaning the area of the patient's skin to be treated prior to application of the topical composition of the present invention; (ii) applying a moisturizer composition the area of the patient's skin to be treated prior to or after application of the topical composition of the present invention; (iii) applying a sunscreen or UV protection composition to the area of the patient's skin to be treated after application of the topical composition of the present invention; and (iv) any combination of (i), (ii) and (iii).

Although any methods and materials similar or equivalent to those described herein can also be used in the practice of the present invention, the preferred methods and materials are now described. All publications mentioned herein are incorporated herein by reference to disclose and describe the methods and/or materials in connection with which the publications are cited.

The following examples are provided by way of illustration only and are by no means intended to be limiting.

### Example 1

Topical compositions in accordance with the present invention may be prepared having the following ingredients with the proviso that the topical compositions comprise at least neferine, liensinine, isoliensine or a combination thereof wherein the amount of neferine, liensinine, isoliensine is at least 0.0005 wt %, preferably at least 0.00075 wt % and most preferably at least 0.001 wt %:

| | WEIGHT PERCENT | | |
|---|---|---|---|
| INGREDIENT | Preferred | More Preferred | Most Preferred |
| Neferine | 0-1.0 | 0-0.50 | 0-0.10 |
| Liensinine | 0-1.0 | 0-0.50 | 0-0.10 |
| Isoliensine | 0-1.0 | 0-0.50 | 0-0.10 |
| Tranexamic acid | 0.05-15.0 | 0.1-12.50 | 1.0-10.0 |
| Additional Melanin | 0-15.0 | 0.1-12.50 | 1.0-10.00 |
| Inhibiting Agent | | | |
| Antioxidant | 0.0005-10.0 | 0.0007-7.50 | 0.001-5.0 |
| Anti-Inflammatory | 0-5.0 | 0.0007-2.50 | 0.001-1.0 |
| Agent | | | |
| Exfoliant | 0-5.0 | 0.0007-2.50 | 0.001-1.0 |
| Skin Conditioning | 0-15.0 | 0.0007-10.0 | 0.001-7.5 |
| Agent | | | |

The topical compositions summarized in the above table may be a solution, suspension, dispersion, emulsion, gel, cream, lotion, ointment or serum and further comprise conventional topical/cosmetic carriers and excipients such as one or more emollients, chelating agents, preservatives, aesthetic agents, pH adjusting agents, water, organic solvents, surfactants, emulsifiers, sunscreen agents and combinations of the foregoing.

In certain embodiments of the topical compositions summarized in the above table, the neferine and liensinine are present and are obtained from a botanical source, preferably a Nelumbo nucifera germ extract wherein the concentration of neferine and liensinine are each greater than 0.05%, preferably greater than 0.075% and more preferably greater than 0.1%.

In certain embodiments of the topical compositions summarized in the above table, the additional melanin inhibiting agent is present and is selected from the group consisting of resorcinol, phenylethyl resorcinol, tri- and tetra-peptides (i.e., such as tetrapeptide-30), kojic diplamitate (aka hexadecanoic acid), vitamin C and derivatives thereof such as tetrahexydecyl ascorbate, plankton extracts, sclareolide and combinations thereof.

In certain embodiments of the topical compositions summarized in the above table, the antioxidant is present and is selected from the group consisting of niacinamide, Vitis vinifera flower cell extract, tocopherol acetate, acetyl tetrapeptide-2, lutein (aka xanthophyll), propyl gallate, hydroxyacetaphenone, sodium bisulfite, diethylhexyl syringylidenemalonate, BHA, BHT, Citrullus lanatus (watermelon) fruit extract, Chamomilla recutita (matricaria) flower extract or combinations thereof.

In certain embodiments of the topical compositions summarized in the above table, the anti-inflammatory agent is present and is selected from the group consisting of dipotassium glycyrrhizate, bisabolol, allantoin, aloe leaf extracts, panthenol, Chamomilla recutita (matricaria) flower extract, Avena sativa (oat) bran extract, or combinations thereof.

In certain embodiments of the topical compositions summarized in the above table, the skin conditioning agent is present and is selected from the group consisting of glycerin, coconut alkanes, Butyrospermum parkii (shea) butter, ethylhexyl olivate, polyisobutene, squalene, cetearyl alcohol, arginine, trehalose, coco-caprylate/caprate, ethylhexylglycerin, sucrose, caprylic/capric triglyceride, sodium hyaluronate, hyaluronic acid, sorbitan isostearate, cyclopentasiloxane, PEG-10 dimethicone, dimethicone/vinyl dimethicone crosspolymer, hydrated silica, ceramide, silica, Dunaliella salina extract, caprylyl glycol, 1-2-hexanediol, aloe barbadensis leaf extract, butylene glycol, panthenol, lens esculenta (lentil) fruit extract, sodium PCA, Laminaria digitata extract, Artemisia vulgaris extract, isopentyldiol, betaine, Hamamelis virginiana (witch hazel) water, Avena sativa (oat) bran extract or combinations thereof.

### Example 2

Topical compositions in accordance with the present invention and particularly topical serum are prepared having the following composition:

| | Weight Percent | | |
|---|---|---|---|
| Ingredient | Preferred | More Preferred | Most Preferred |
| Water | 50-90 | 60-85 | 65-80 |
| Tranexamic acid | 1-15 | 2-12 | 3-9 |
| Nelumbo Nucifera Germ | 0.01-2 | 0.05-1 | 0.075-0.5 |
| Extract | | | |
| Niacinamide | 0.1-10 | 0.25-7.5 | 0.5-5 |
| Phytic Acid | 0.1-7.5 | 0.5-5 | 0.75-2.5 |
| Phenylethyl Resorcinol | 0.1-7.5 | 0.5-5 | 0.75-2.5 |
| Vitis Vinifera (Grape) | 0-1.0 | 0.01-0.80 | 0.1-0.7 |
| Flower Cell Extract | | | |
| Sclareolide | 0.01-1 | 0.05-0.75 | 0.075-0.5 |
| Sodium Hyaluronate | 0.001-1 | 0.005-0.75 | 0.0075-0.5 |
| Squalane | 0.01-2 | 0.05-1.5 | 0.1-1.0 |
| Hydroxyacetophenone | 0.01-2 | 0.05-1.5 | 0.1-1.0 |
| Diethyl hexyl | 0.01-2 | 0.05-1 | 0.07-0.5 |
| Syringyldenemalonate | | | |
| Xanthophyll | 0.001-0.5 | 0.005-0.1 | 0.01-0.08 |

The Nelumbo nucifera germ extract in the above table has a neferine concentration of about 0.1% to about 0.2% based on the total weight of the Nelumbo nucifera germ extract and a liensinine concentration of about 0.1% to about 0.2% based on the total weight of the Nelumbo nucifera germ extract.

The topical compositions described in the above table may further comprise glycerin, coconut alkanes, polyacrylate-13, C₁₀-C₁₈ triglycerides, dicaprylyl carbonate, C₁₂-C₁₅ alkyl benzoate, Butyrospermum parkii (shea) butter, ethylhexyl olivate, phenoxyethanol, polyisobutene, propanediol, cetearyl alcohol, arginine, arachidyl alcohol, mica, behenyl alcohol, trehalose, polysorbate 80, titanium dioxide, coco-caprylate/caprate, xanthan gum, arachidyl glucoside, disodium EDTA, ethylhexylglycerin, lecithin, sclerotium gum, sucrose, sodium stearoyl glutamate, pullulan, caprylic/capric triglyceride, 1,2-hexanediol, tin oxide, caprylhydroxamic acid, sorbitan isostearate silica and combinations thereof.

### Example 3

Topical compositions in accordance with the present invention and particularly topical liquids are prepared having the following composition:

| | Weight Percent | | |
|---|---|---|---|
| Ingredient | Preferred | More Preferred | Most Preferred |
| Cyclopentasiloxane | 10-65 | 15-60 | 20-55 |
| Dimethicone | 1-30 | 2.5-25 | 5-20 |
| Crosspolymer | | | |
| Water | 1-30 | 2.5-25 | 5-20 |
| Caprylic/Capric | 1-30 | 2.5-25 | 5-20 |
| Trigylceride | | | |
| Niacinamide | 0.5-15 | 0.75-10 | 1-7.5 |
| Tranexamic Acid | 0.5-15 | 0.75-10 | 1-6 |
| Glycolic Acid | 0.5-15 | 0.75-10 | 1-6 |
| Tetrahexyldecyl | 0.1-10 | 0.5-7 | 0.75-5 |
| Ascorbate | | | |
| Kojic Dipalmitate | 0.05-5 | 0.1-3 | 0.5-2 |
| Phenylethyl Resorcinol | 0.05-5 | 0.1-3 | 0.5-2 |
| Diethylhexyl | 0.01-1.5 | 0.05-1 | 0.01-0.075 |
| Syringylidenemalonate | | | |
| Retinol | 0-1.5 | 0-1 | 0-0.075 |
| Bisabolol | 0.01-1.5 | 0.05-1 | 0.01-0.075 |
| Tocopheryl Acetate | 0.01-1.5 | 0.05-1 | 0.01-0.075 |
| Allantoin | 0.01-1.5 | 0.05-1 | 0.01-0.075 |
| Nelumbo Nucifera | 0.01-1.5 | 0.05-1 | 0.01-0.075 |
| Germ Extract | | | |
| Dipotassium | 0.01-1.5 | 0.05-1 | 0.01-0.075 |
| Glycyrrhizate | | | |
| Squalane | 0.01-1.5 | 0.05-1 | 0.01-0.075 |
| Ceramide | 0.001-0.7 | 0.005-0.5 | 0.007-0.1 |
| Acetyl Tetrapeptide-2 | 0.00001-0.01 | 0.00005-0.005 | 0.0001-0.001 |

The Nelumbo nucifera germ extract in the above table has a neferine concentration of about 0.1% to about 0.2% based on the total weight of the Nelumbo nucifera germ extract and a liensinine concentration of about 0.1% to about 0.2% based on the total weight of the Nelumbo nucifera germ extract.

The topical compositions described in the above table may further comprise glycerin, PEG-10 dimethicone, dimethicone, PEG/PPG-18/18 dimethicone, aminomethyl propanediol, dimethicone/vinyl dimethicone crosspolymer, titanium dioxide, Propanediol, stearalkonium hectorite, sodium bisulfite, sodium surfactin, xanthan gum, hydrated silica, polysorbate 20, BHT, phenoxyethanol, propylene carbonate, cetyl palmitate, laureth-23, sodium hydroxide, trideceth-6 phosphate, silica, isoceteth-10, ethylhexylglycerin, Dunaliella salina extract, caprylyl glycol, 1,2-hexanediol, capryihydroxamic acid and combinations thereof.

### Example 4

Topical compositions in accordance with the present invention and particularly topical serum are prepared having the following composition:

| | Weight Percent | | |
|---|---|---|---|
| Ingredient | Preferred | More Preferred | Most Preferred |
| Water | 65-95 | 70-90 | 75-87 |
| Aloe Barbadensis Leaf | 0.1-15 | 0.5-10 | 1-8 |
| Extract | | | |
| Tranexamic acid | 0.5-12 | 0.75-9 | 1-6 |
| Glycolic Acid | 0.05-7.5 | 0.1-5 | 0.5-2.5 |
| Phytic Acid | 0.01-2.5 | 0.05-2 | 0.1-1 |
| Citrullus Lanatus | 0.005-2 | 0.0075-1.5 | 0.001-1 |
| (Watermelon) Fruit | | | |
| Extract | | | |
| Panthenol | 0.005-1.5 | 0.0075-1 | 0.001-0.75 |
| Lens Esculenta (Lentil) | 0.005-1.5 | 0.0075-1 | 0.001-0.75 |
| Fruit Extract | | | |
| Pyrus Malus (Apple) | 0.005-1.5 | 0.0075-1 | 0.001-0.75 |
| Fruit Extract | | | |
| Niacinamide | 0.005-1 | 0.0075-0.75 | 0.001-0.5 |
| Allantoin | 0.001-0.75 | 0.005-0.5 | 0.01-0.1 |
| Sodium Hyaluronate | 0.001-0.75 | 0.005-0.5 | 0.01-0.1 |
| Sodium PCA | 0.001-0.5 | 0.005-0.25 | 0.0075-0.1 |
| Nelumbo Nucifera Germ | 0.001-0.3 | 0.005-0.2 | 0.0075-0.1 |
| Extract | | | |
| Laminaria Digitata | 0.001-0.3 | 0.005-0.2 | 0.0075-0.1 |
| Extract | | | |
| Artemisia Vulgaris | 0.001-0.3 | 0.005-0.2 | 0.0075-0.1 |
| Extract | | | |
| Chamomilla Recutita | 0.0001-0.05 | 0.0005-0.01 | 0.0007-0.007 |
| (Matricaria) Flower | | | |
| Extract | | | |

The Nelumbo nucifera germ extract in the above table has a neferine concentration of about 0.1% to about 0.2% based on the total weight of the Nelumbo nucifera germ extract and a liensinine concentration of about 0.1% to about 0.2% based on the total weight of the Nelumbo nucifera germ extract.

Topical compositions described in the above table may further comprise glycerin, butylene glycol, PEG-12 dimethicone, sodium benzoate, phenoxyethanol, carpylyl glycol, disodium EDTA, chlorphenesin, sodium lactate, propanediol, isopentyldiol, polysorbate 20, 1,2-hexanedio, caprylhydroxamic acid, silica, potassium sorbate and combinations thereof.

### Example 5

Topical compositions in accordance with the present invention and particularly topical liquids for use with pads or wipes are prepared having the following composition:

| | Weight Percent | | |
|---|---|---|---|
| Ingredient | Preferred | More Preferred | Most Preferred |
| Water | 45-90 | 50-85 | 55-80 |
| Hamamelis Virginiana | 1-25 | 2.5-20 | 5-15 |
| (Witch Hazel) Water | | | |
| Betaine | 0.1-10 | 0.5-7 | 0.75-5 |
| Glycolic Acid | 1-25 | 2.5-20 | 5-15 |
| Niacinamide | 0.1-7.5 | 0.5-5 | 0.75-3 |
| Tranexamic Acid | 0.1-7.5 | 0.5-5 | 0.75-3 |
| Dipotassium | 0.01-2 | 0.05-1 | 0.075-0.75 |
| glycyrrhizinate | | | |
| Nelumbo Nucifera Germ | 0.001-0.3 | 0.005-0.2 | 0.0075-0.1 |
| Extract | | | |

The Nelumbo nucifera germ extract in the above table has a neferine concentration of about 0.1% to about 0.2% based on the total weight of the Nelumbo nucifera germ extract and a liensinine concentration of about 0.1% to about 0.2% based on the total weight of the Nelumbo nucifera germ extract.

Topical compositions described in the above table may further comprise sodium gluconate, polysorbate 20, benzoic acid, Avena sativa (oat) bran extract, sodium benzoate, potassium sorbate, Propanediol, caprylhydroxamic acid, 1,2 hexanediol, phenoxyethanol, ethylhexylglycerin, sodium hydroxide and combinations thereof.

### Example 6

A split-face, double-blind, randomized, 12-week clinical study comparing a topical composition as described in Example 2 and a 4% hydroquinone topical cream was conducted. Participants with moderate to severe facial hyperpigmentation, including melasma, applied the composition of Example 2 to a randomly assigned facial side and the 4% hydroquinone topical cream, (“4% HQ”) to the opposite facial side twice a day (AM/PM) for twelve (12) weeks. A basic skincare regimen was also applied to the entire face during the study comprising twice daily washing with SKINMEDICA Facial Cleanser [AM/PM], twice daily application of SKINMEDICA Ultra Sheer Moisturizer [AM/PM] and once daily application of SKINMEDICA Essential Defense Mineral Shield Broad Spectrum SPF 35 Sunscreen [AM only]). Participants were instructed to avoid extended periods of sun exposure and to wear protective clothing. The 4% HQ topical cream was commercially available from Westminster Pharmaceuticals and each gram contained 40 mg of hydroquinone in a base comprising glyceryl monostearate, mineral oil, PEG-25 propylene glycol stearate, polyoxl-40 stearate, propylene glycol, propylparaben, purified water, sodium metabisulfae, squalene and stearic acid.

Included participants were healthy females 30-65 years of age with a Fitzpatrick Skin Type (“FST”) I-VI, with moderate to severe hyperpigmentation on both sides of the face (scores of 4-9 per modified Griffiths scale: 0=none [best possible condition]; 9=severe [worst possible condition]) and with ≤1 difference in scores between facial sides were enrolled. The study design required enrollment of at least 25 participants in each of the following ethnic/racial groups: Asian, Black/African America, Hispanic, and White/Caucasian. Furthermore, at least 30 participants with epidermal facial melasma, a score ≥2 on the Melasma Severity Rating Scale and FST III or higher were required. Participants with a history of skin cancer within the past 5 years or currently using oral or topical prescription medications for acne were excluded from the study, as were those using over-the-counter retinol-containing anti-wrinkle, skin-lightening, or other topical or systemic products within the previous 4 weeks.

The topical composition of Example 2 comprised water, tranexamic acid, niacinamide, glycerin, coconut alkanes, phytic acid, polacrylate-13, phenylethyl resorcinol, Ciois triglycerides, dicaprylyl carbonate, C₁₂₋₁₅ alkyl benzoate, shea butter, ethylhexyl olivate, phenoxyethanol, polyisobutene, squalane, hydroxyacetophenone, propanediol, cetearyl alcohol, arginine, arachidyl alcohol, mica, Vitis vinifera (grape) flower cell extract, trehalose, diethylhexyl syringylidenemalonate, polysorbate 20, titanium dioxide, coco-caprylate/caprate, xanthan gum, arachidyl glucoside, Nelumbo nucifera germ extract, sclareolide, disodium EDTA, ethylhexylglyceri, lecithin, sclerotium gum, sucrose, sodium stearoyl glutamate, pullulan, xanthophyll, caprylic/capric triglyceride, sodium hyaluronate, 1,2-hexanediol, tin oxide, caprylhydroxamic acid, sorbitan isostearate and silica.

A total of 113 female participants, including 44 in the melasma subgroup, with FST 4-VI were enrolled in the 12-week clinical study, with approximately equivalent representation of each race and ethnic subgroup: Asian, n=25 (22.1%); Black/African American, n=31 (27.4%); Hispanic, n=25 (22.1%); White/Caucasian, n=32 (28.3%). Nearly half (46.9%) of participants had FST IV-VI. The melasma subgroup also had approximately equal racial/ethnic representation: Asian, n=11 (25.0%); Black/African American, n=9 (20.5%); Hispanic, n=12 (27.3%); and White/Caucasian, n=12 (27.3%); and included FST I32-VI. The participant disposition and baseline characteristics are shown in the following table:

| | All Participants | Melasma Subgroup |
|---|---|---|
| Characteristic | (N = 113) | (n = 44) |
| Age, years | | |
| Mean (SD) | 52.6 (8.0) | 49.8 (7.7) |
| Median (minimum, | 53.0 (31.0, 65.0) | 51.0 (31.0, 63.0) |
| maximum) | | |
| Sex, female, n (%) | 113 (100) | 44 (100) |
| Race and ethnic subgroup, | | |
| n (%) | | |
| Asian | 25 (22.1) | 11 (25.0) |
| Black or African American | 31 (27.4) | 9 (20.5) |
| Hispanic | 25 (22.1) | 12 (27.3) |
| White or Caucasian | 32 (28.3) | 12 (27.3) |
| Fitzpatrick skin type, n (%) | | |
| I | 1 (0.9) | 0 |
| II | 11 (9.7) | 0 |
| III | 48 (42.5) | 26 (59.1) |
| IV | 25 (22.1) | 11 (25.0) |
| V | 24 (21.2) | 6 (13.6) |
| VI | 4 (3.5) | 1 (2.3) |
| Hyperpigmentation score at | Ex. 2 side, 4.95 | Ex. 2 side, 5.16, |
| baseline, mean | 4% HQ side, 4.95 | 4% HQ side, 5.17 |
| mMASI grading at baseline, | NA | EX. 2 side, 4.50, |
| mean | | 4% HQ, 4.42 |

Study visits occurred at baseline and weeks 2, 4, 8, and 12. The following assessments were conducted and results obtained during the study:

(i) Hyperpigmentation and Skin Tone Evenness

Investigator-assessed clinical grading of hyperpigmentation and skin tone evenness was performed separately on each side of the face at each study visit and scored on a modified Griffiths scale as none (score of 0, best possible condition), mild (1 to 3), moderate (4 to 6), and severe (7 to 9, worst possible condition). Griffiths et al., “A Photonumeric Scale for the Assessment of Cutaneous Photodamage,” Arch Dermatol. 1992; 128(3):347-351.

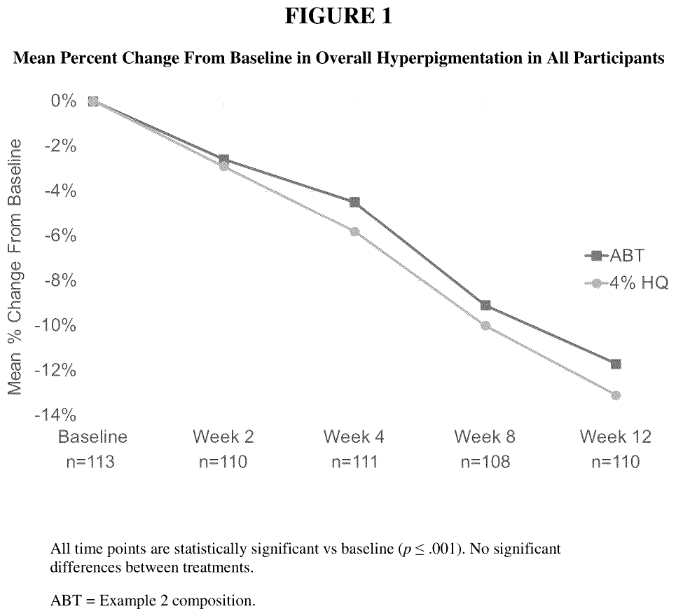

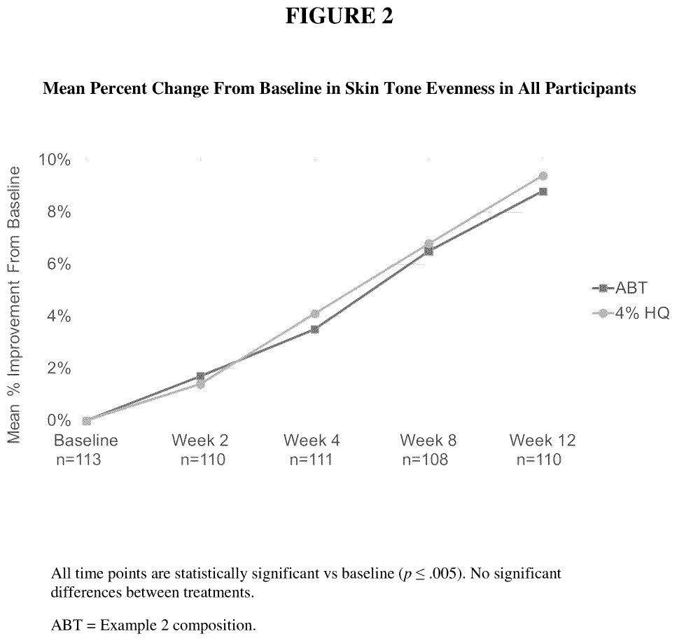

For both Example 2 and 4% HQ, statistically significant improvements versus baseline were observed as early as week 2 for overall hyperpigmentation and skin tone evenness with continuing significant improvements at each follow-up visit through week 12 (p≤0.005). The mean percentage change for baseline in overall hyperpigmentation in all participants is shown in FIG. 1 and the mean percent change from baseline in skin tone evenness in all participants is shown in FIG. 2. The data shows the efficacy of the compositions in accordance with Example 2 are equivalent to that of 4% HQ, with no significant differences between treatments.

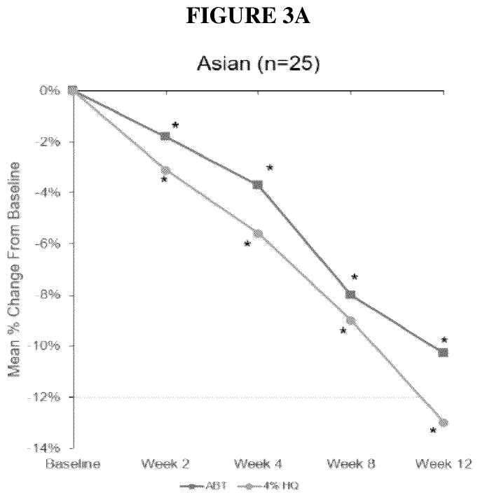

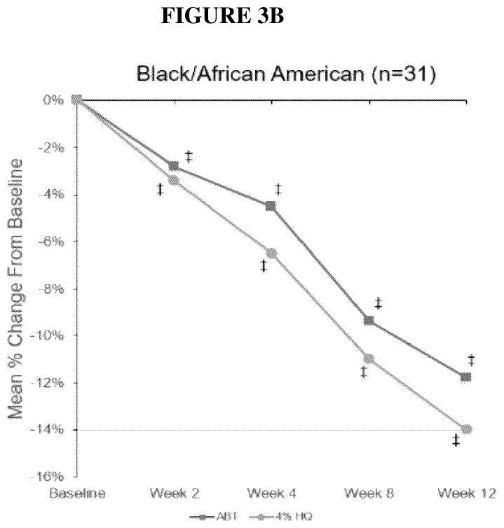

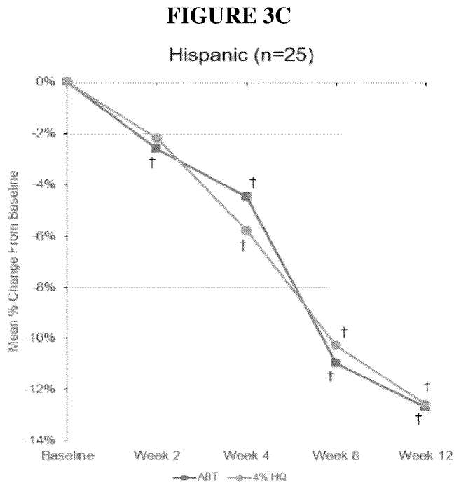

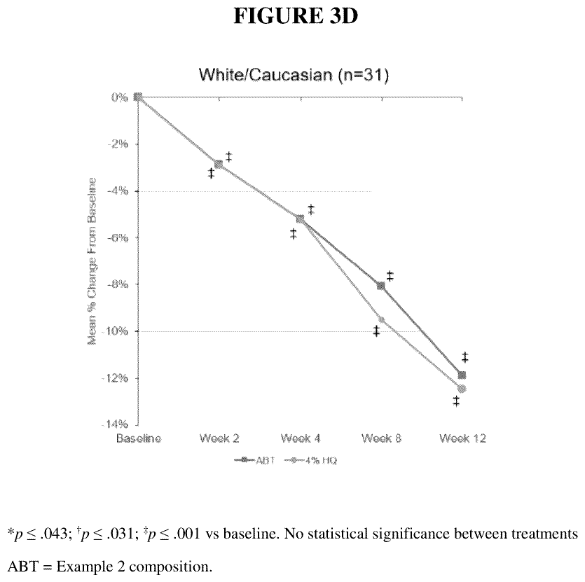

Results were similar across all ethnicities, with significant improvements versus baseline in overall hyperpigmentation observed at week 2 for both Example 2 and 4% HQ (week 4 for the Hispanic subgroup) and continuing through week 12 (p≤0.043 across all ethnicities). Similarly, skin tone evenness was significantly improved with Example 2 and 4% HQ starting at week 2 in Black/African American participants and at week 4 in Asian, Hispanic, and White/Caucasian participants through week 12 (p≤0.031 across all ethnicities). The mean percentage change for baseline in overall hyperpigmentation in the race/ethnic subgroups is shown in FIGS. 3A-3D. The data shows the compositions in accordance with Example 2 achieved comparable results to 4% HQ across all ethnicities, with no significant differences between treatments.

(ii) Modified Melasma Area and Severity Index

Melasma severity was assessed using the modified Melasma Area and Severity Index (mMASI) [See, Pandya, et al., “Reliability Assessment and Validation of the Melasma Area and Severity Index (MASI) and a New Modified MASI Scoring Method,” J Am Acad Dermatol 2011; 64:78-83, 83.e1-2] for the melasma subgroup only at each study visit, with left and right sides assessed separately.

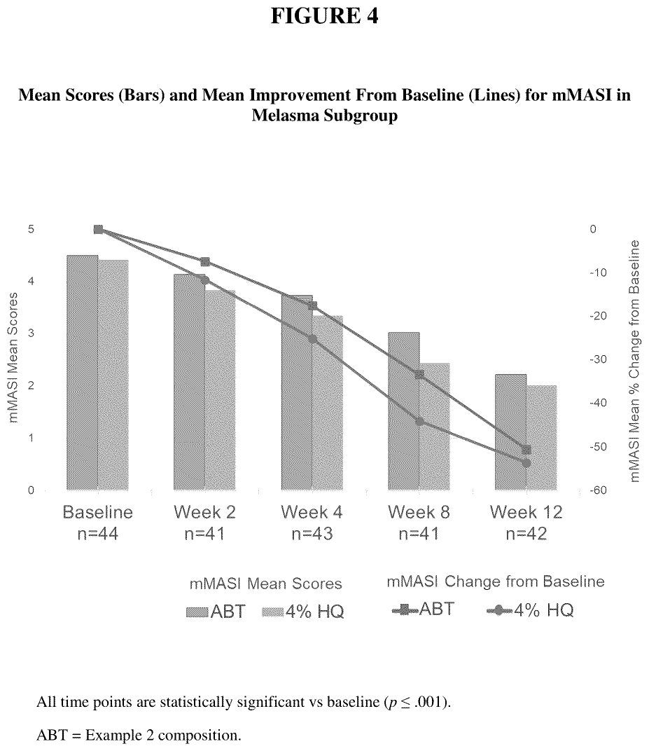

Compared with baseline, both Example 2 and 4% HQ significantly improved melasma severity as measured using mMASI at each time point from week 2 through week 12 in the melasma subgroup (p≤0.011), with no significant differences between treatments. FIG. 4 shows the mean scores and mean improvement from baseline for mMASI in the melasma subgroup.

(iii) Melasma Quality of Life Questionnaire

A validated Melasma Quality of Life Questionnaire (MelasQoL) was used to measure the emotional and psychological impact of melasma in the melasma subgroup at each study visit. Higher scores indicate worse melasma-related health-related quality of life. MelasQoL is validated for full-face melasma; as such, each treatment side was not evaluated separately.

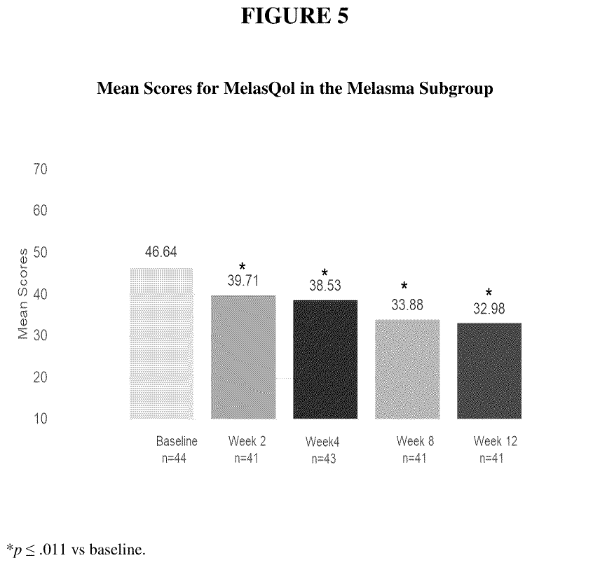

Participant assessment of emotional and psychological effects of melasma using the MelasQoL questionnaire was significantly improved in the melasma subgroup at each time point throughout the study (p≤0.011 at all time points). The mean scores are shown in FIG. 5.

(iv) Self-Assessment Questionnaires

Participants completed self-assessment questionnaires at weeks 2, 4, 8, and 12 on self-perceived efficacy, test product attributes, overall improvement, overall satisfaction, and face side (i.e., test product) preference, all evaluated separately on left and right sides of face.

Self-assessed overall improvement in skin condition was comparable between treatments across all study visits, with 95% and 94% of participants reporting at least a ˜25% overall improvement in their skin condition with Example 2 and 4% HQ, respectively, at week 12. Among all participants, overall satisfaction with results on each facial side was comparable between the two treatments across all visits. More participants, both in the overall group and the melasma subgroup, preferred the Example 2 composition over 4% HQ across all visits from week 2 through week 12.

At week 12, self-reported efficacy across a range of efficacy parameters was high and comparable between Example 2 (range 75% to 84%) and 4% HQ (range 76% to 86%. At week 12, Example 2 was highly rated (>90%) and preferred over 4% HQ regarding multiple product attributes, including easy to apply, spreads easily on skin, absorbed easily in skin, works well with other products in my daily regimen, and pleasant texture. A summary of the self-reported data at 12 weeks in all participants is provided below.

| | Example 2 | 4% HQ |
|---|---|---|
| Improved the overall appearance of my skin | 84% | 86% |
| Improved the overall condition of my skin | 81% | 81% |
| Makes my skin feel smooth and soft | 84% | 84% |
| Made my complexion more radiant | 79% | 81% |
| Improved the evenness of my skin tone | 84% | 85% |
| Faded the age spots on my skin | 80% | 77% |
| Helped prevent new dark spots | 75% | 76% |
| Makes my skin look less dull | 84% | 78% |
| Improved the look of sun damage | 80% | 81% |
| Makes me feel happier about how I look | 77% | 78% |
| Improved the overall tone and clarity of my skin | 83% | 84% |
| Improved current and helps prevent uneven pigment | 75% | 76% |
| in my skin | | |

(v) Tolerability Parameters

Tolerability evaluations, including investigator-assessed objective irritation parameters (erythema, edema, and dryness) and participant-reported subjective irritation parameters (burning, stinging, and itching), were performed at each study visit on a 4-point scale, where 0=none, 1=mild, 2=moderate, and 3=severe.

Example 2 and 4% HQ were well tolerated in both the overall group (n=113) and the melasma subgroup (n=44) at all follow-up visits. Mean scores for all tolerability parameters remained similar to baseline scores (below “mild”) at all study visits. No serious treatment-emergent adverse events (AEs) were reported and no pattern of treatment-emergent AEs was observed. Treatment-related AEs (TRAEs) were reported by three participants on the Example 2 treated side and by 4 participants on the 4% HQ-treated side. TRAEs on the Example 2 treated side included erythema, pain, dryness, edema, itching, rash, and acne; TRAEs on the 4% HQ-treated side included erythema, dryness, itching, rash, burning, and acne. Two participants discontinued the study due to AEs (itching, erythema, pain, dryness, edema, and rash) experienced on both treatment sides. At the end of the study, TRAEs had resolved in all participants.

Participants were also photographed using VISIA CR photo station (Canfield Imaging Systems, Fairfield, NJ, USA) with a Canon Mark II digital SLR camera (Canon Incorporated, Tokyo, Japan) at each study visit.

All statistical analyses were conducted on the intent-to-treat population, defined as all participants who received treatment and participated in at least 1 post-baseline evaluation. For efficacy parameters, change from baseline was used to compare treatments, and a descriptive statistical summary was developed (e.g., mean, standard deviation [SD]). The testing hypothesis was that the change from baseline is equal between the 2 treatments, using Wilcoxon signed-rank test for clinical grading of efficacy parameters, mMASI, self-assessment questionnaires, and tolerability evaluations, and paired t test for MelasQoL. All statistical tests were 2 sided with significance set at α=0.05 (unless otherwise specified). Statistical analyses were performed using SAS software version 9.4 (SAS Statistical Institute, Cary, NC, USA).

The data provided in this Example demonstrates that the compositions in accordance with Example 2, an HQ-free, multimodal pigment-correcting serum, is an effective and well-tolerated topical treatment for moderate to severe facial hyperpigmentation, including melasma. Compositions in accordance with Example 2 are effective in reducing the severity of overall hyperpigmentation and melasma as early as 2 weeks, with continued improvement through week 12 and a high rate of treatment satisfaction. In addition, compositions in accordance with Example 2 show efficacy in a broad range of racial/ethnic subgroups, as well as in participants with FST I-VI.

### Example 7

A 12-week single-center study to assess the efficacy and tolerability of topical composition as described in Example 3 for improving mild to moderate post-inflammatory hyperpigmentation (PIH) or solar lentigines was conducted. Participants with mild to moderate facial dark spots applied the composition of Example 3, once a day in the evening to only the dark facial spots and avoiding normal skin for 12 weeks. A basic skincare regimen was also applied to the entire face during the study comprising twice daily washing with SKINMEDICA Facial Cleanser [AM/PM], twice daily application of SKINMEDICA Ultra Sheer Moisturizer [AM/PM] and once daily application of SKINMEDICA Essential Defense Mineral Shield Broad Spectrum SPF 35 Sunscreen [AM only]). Participants were instructed to avoid application of any topical moisturizing products or other treatments to the face for at least 3 days prior to visit 1.

Included participants were healthy females 25-65 years of age with mild to moderate facial dark spots (score of 3-6 on a modified Griffiths scale). The PIH subgroup included female participants with a Fitzpatrick Skin Type (“FST”) III-VI and a least 1 PIH spot ≥3 mm and at least 75% Black/African American, Asian or Hispanic Caucasian participants. The solar lentigines subgroup included female participants with FST I-III and at least 1 age spot/solar lentigines ≥3 mm on the face and at least 50% Asian or Hispanic Caucasian participants. All study participants were required to have not used facial treatments in the previous 6 months. Participants with a history of skin cancer within the past 5 years, known allergies to facial skin products, breastfeeding, pregnant or planning to become pregnant, current or previous use within the last 3 months of an oral or topical prescription medication for acne, retinol-containing products within 4 weeks, or other products known to affect skin aging or dyschromia within 2 weeks were excluded from the study.

The topical composition of Example 3 comprised water, cyclopentasiloxane, dimethicone crosspolymer, caprylic/capric triglyceride, niacinamide, glycerin, glycolic acid, tetrahexyldecyl ascorbate, PEG-10 dimethicone, kojic dipalmitate, dimethicone, PEG/PPG-18/18 dimethicone, phenylethyl resorcinol, aminomethyl propanediol, dimethicone/vinyl dimethicone cross polymer, titanium dioxide, propanediol, stearalkonium hectorite, sodium bisulfite, sodium surfactin, xanthan gum, hydrated silica, diethylhexylsyringylidenemalonate, retinol, polysorbate 20, butylated hydroxytoluene, bisabol, tocopheryl acetate, phenoxyethanol, allantoin, Nelumbo nucifera germ extract, propylene carbonate, dipotassium glycyrrhizate, cetyl palmitate, laureth-23, squalene, ceramide NP, sodium hydroxide, trideceth-6 phosphate, silica, isoceteth, ethylhexylglycerin, Dunaliella salina extract, caprylyl glycol, 1,2-hexanediol, acetyl tetrapeptide-2 and caprylhydroxamic acid.

A total of 41 female participants, aged 25-64, participated in the study. The PIH group consisted of 19 female participants who were predominatly Black/African American (52.6%) and Hispanic (36.8%), with FST IV-V. The solar lentigines group consisted of 22 females who were predominantly White/Caucasian (50.0%), Hispanic (22.7%), and Asian (18.2%), with FST I-IV The participant disposition and baseline characteristics are shown in the following table:

| | | Solar | All |
|---|---|---|---|
| | PIH | Lentigines | Participants |
| | (n = 19) | (n = 22) | (N = 41) |
| Race and Ethnicity, n (%) | | | |
| White/Caucasian | 1 (5.3) | 11 (50.0) | 12 (29.3) |
| Hispanic | 7 (36.8) | 5 (22.7) | 12 (29.3) |
| Black/African American | 10 (52.6) | 0 | 10 (24.4) |
| Asian | 1 (5.3) | 4 (18.2) | 5 (12.2) |
| Native American/Alaska Native | 0 | 1 (4.5) | 1 (2.4) |
| Asian/Caucasian | 0 | 1 (4.5) | 1 (2.4) |
| Fitzpatrick Skin Type, n (%) | | | |
| I | 0 | 1 (4.5) | 1 (2.4) |
| II | 0 | 9 (40.9) | 9 (22.0) |
| III | 0 | 10 (45.5) | 10 (24.4) |
| IV | 10 (52.6) | 2 (9.1) | 12 (29.3) |
| V | 9 (47.4) | 0 | 9 (22.0) |

Study visits occurred at baseline and weeks 2, 4, 8, and 12. Investigators performed clinical grading of the full face at baseline and Weeks 2, 4, 8, and 12 using the modified Griffiths 10-point scale for overall hyperpigmentation, skin tone evenness, and dark spot intensity, contrast, and size. Lower scores on this scale indicate improvement (0=none, 1-3=mild, 4-6=moderate, 7-9=severe). Standardized digital photographs were taken of the participants' full faces (left, front, right) using a VISIA-CR photo station with a Canon Mark II digital SLR camera (Canfield Scientific, Parsippany, NJ, USA). Quantitative analysis of target dark spot skin brightness (L*) was evaluated using VAESTRO Image Analysis Software (Canfield Scientific).

Investigators performed clinical grading for tolerability using a 4-point scale (0=none, 1=mild, 2=moderate, 3=severe) by assessing signs of erythema, edema, and dryness. Using the same scale, participants reported the degree of burning, stinging, itching, and peeling globally on their face.

Participant self-assessments were made at Weeks 1, 2, 4, 8, and 12. Participants completed a sponsor-provided self-assessment questionnaire on treatment satisfaction, the effect of immediate and continued use, skin texture, and application experience.

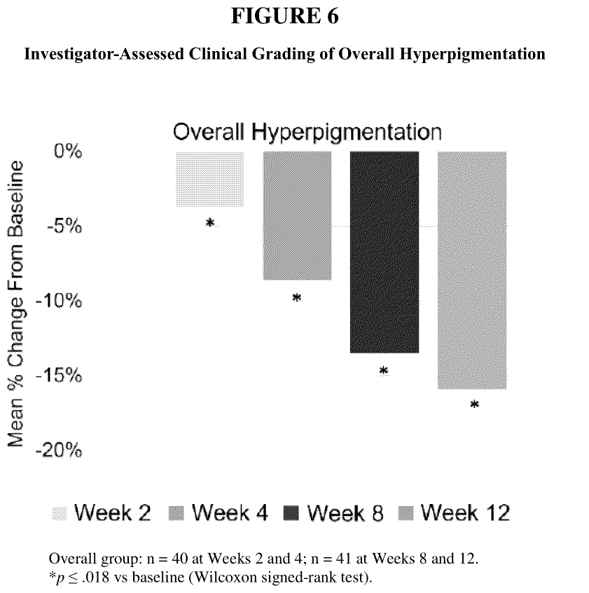

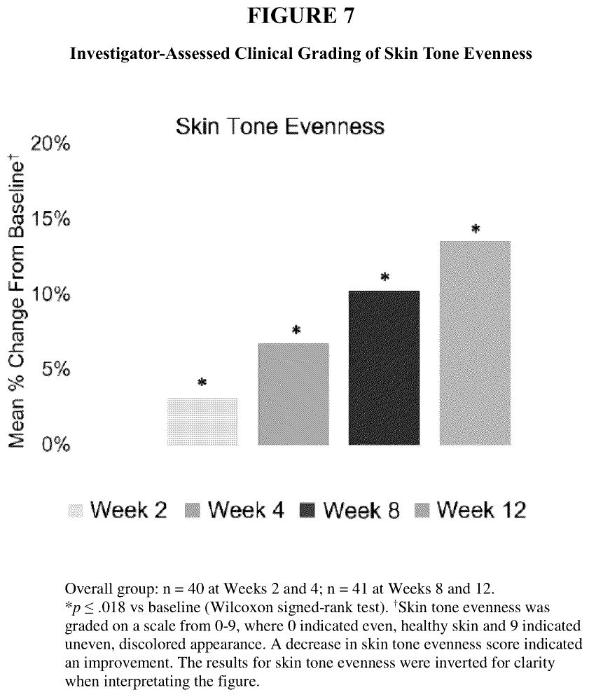

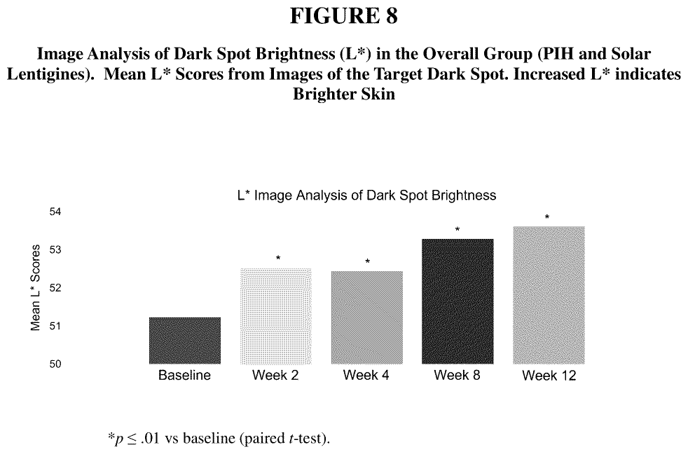

As shown in FIGS. 6 and 7, the application of the composition described in Example 3 once daily for 12 weeks resulted in a significant reduction in overall hyperpigmentation and a significant improvement in skin tone evenness in the overall group (combined PIH and solar lentigines) compared with baseline starting at week 2 and continuing through week 12 (p≤0.018). As shown in FIG. 8, image analysis of target dark spot brightness (L*) showed significant improvement at all visits in the overall group (p≤0.01).

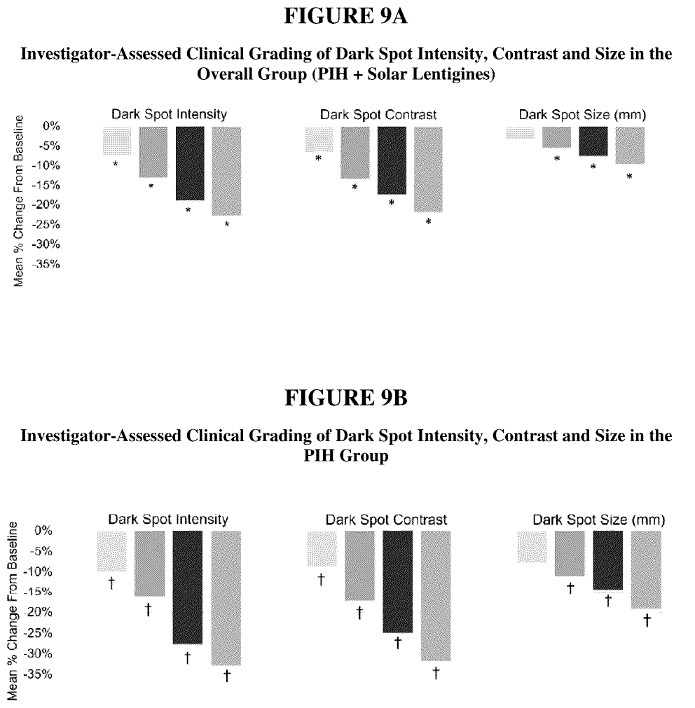

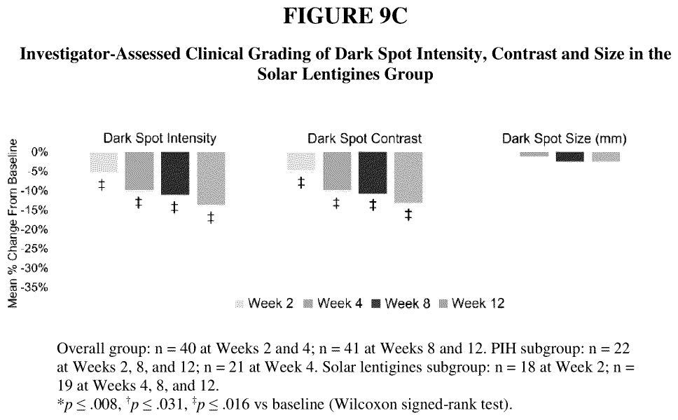

Dark spot intensity and dark spot contrast were significantly reduced with the topical composition of Example 3 at all follow-up visits, and dark spot size at weeks 4, 8, and 12, compared to baseline in the overall group (p≤0.008) (FIG. 9A). Subgroup analysis showed similar results for the PIH subgroup with significant improvements in dark spot intensity and dark spot contrast starting at week 2, and in dark spot size starting at week 4 (p≤0.031) (FIG. 9B). In the solar lentigines subgroup, dark spot intensity and dark spot contrast were significantly reduced at all visits (p≤0.016), and there was no significant change in dark spot size (FIG. 9C).

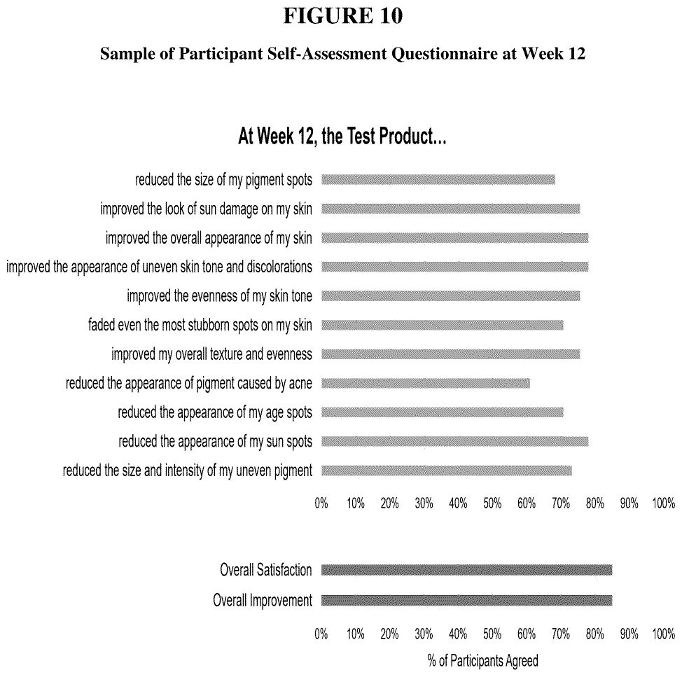

As shown in FIG. 10 participant self-assessment questionnaires showed consistent, high levels of self-perceived effectiveness across the questionnaire topics with the use of topical composition of Example 3 during the 12-week study. Furthermore, 85% of participants saw an overall improvement in their skin condition with the topical composition of Example 3 at week 12, and ≥85% of participants reported overall satisfaction with the results at all time points.

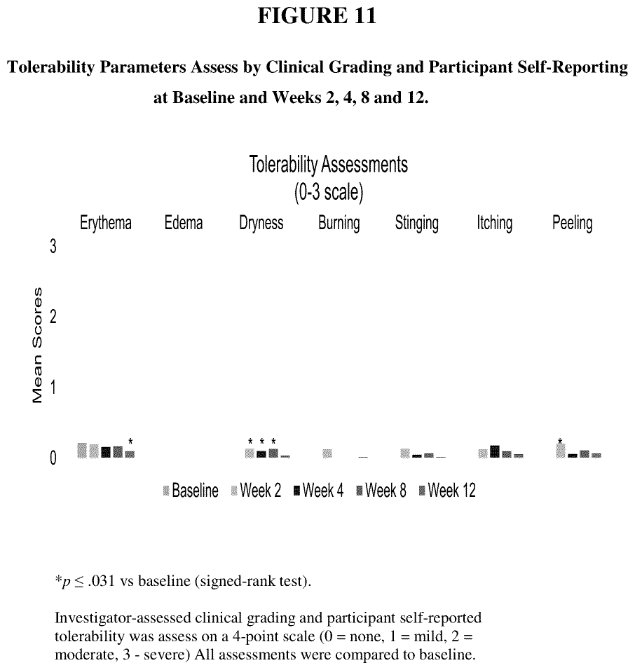

Investigator-assessed clinical grading and participant self-reporting of tolerability parameters (erythema, edema, dryness, burning, stinging, itching, peeling) showed that the topical composition of Example 3 was well tolerated during the 12-week study. As shown in FIG. 11, the occurrence of erythema (week 12), dryness (weeks 2, 4, and 8), and peeling (week 2) were significant compared with baseline; however, mean tolerability scores for all parameters were less than 0.25 (mild) at all study visits. No serious adverse events were reported.

The data provided in this Example demonstrates that the compositions in accordance with Example 3 are an effective and tolerable treatment for hyperpigmentation associated with PIH and solar lentigines. Improvements in facial dark spots are seen as early as week 2 and continued through week 12. The results further demonstrate the compositions in accordance with Example 3 are effective in a broad range of ages, ethnicities, and skin types.

### Example 8

A 12-week single-center study to assess the efficacy and tolerability of topical composition as described in Example 4 when used as part of a diamond tip microdermabrasion treatment and in combination with the topical compositions of Example 2/6 and Example 3/7 for improving mild to severe facial hyperpigmentation, including melisma, post-inflammatory hyperpigmentation (PIH) and dark spots was conducted. Participants with mild to severe facial hyperpigmentation received biweekly in-office SKINMEDICA® DIAMOND GLOW® (diamond tip microdermabrasion) treatments for a total of six in-office treatments with the composition of Example 4. The participants also applied at home the topical composition as described in Example 6 twice a day (AM/PM) for twelve (12) weeks and the topical composition as described in Example 7 once a day (PM) for twelve (12) weeks. A basic skincare regimen was also applied to the entire face during the study comprising twice daily washing with SKINMEDICA Facial Cleanser [AM/PM], twice daily application of SKINMEDICA Ultra Sheer Moisturizer [AM/PM] and once daily application of SKINMEDICA Essential Defense Mineral Shield Broad Spectrum SPF 35 Sunscreen [AM and as needed]). Participants were instructed to avoid application of any topical moisturizing products or other treatments to the face for at least 3 days prior to visit 1.

Included participants were healthy females 18-65 years of age (Fitzpatrick Skin Type [FST] I-VI) with mild to severe facial hyperpigmentation, score of 3-9 on a modified Griffiths 10 point scale; with ≥15 participants with moderate/severe overall hyperpigmentation and 3-5 participants with mild overall hyperpigmentation.

All study participants were required to have not used facial treatments in the previous 6 months. Participants with a history of skin cancer within the past 5 years, known allergies to facial skin products, breastfeeding, pregnant or planning to become pregnant, current or previous use within the last 3 months of an oral or topical prescription medication for acne, retinol-containing products within 4 weeks, or other products known to affect skin aging or dyschromia within 2 weeks were excluded from the study.

The topical composition of Example 4 comprised water, aloe barbadensis leaf extract, tranexamic acid, glycerin, butylene glycol, glycolic acid, phytic acid, PEG-12 dimethicone, sodium benzoate, Citrullus lanatus (watermelon) fruit extract, phenoxyethanol, panthenol, lens esculenta (lentil) fruit extract, pyrus malus (Apple) fruit extract, carpylyl glycol, niacinamide, disodium EDTA, chlorphenesin, sodium lactate, propanediol, allantoin, sodium hyaluronate, sodium pyrrolidone carboxylic acid (“PCA”), Nelumbo nucifera germ extract, Laminaria digitata extract, Artemisia vulgaris extract, isopentyldiol, Chamomilla recutita (Matricaria) flower extract, polysorbate 20, 1,2-hexanediol, caprylhydroxamic acid, silica and potassium sorbate.

A total of 18 female and male subject enrolled in the study and 17 female subjects completed the study. The participants were White/Caucasian (56.0%), Hispanic (6.0%), and Asian (39%), with FST I (2 subjects (11.11%)), FST II (1 subject (5.56%)), FST III (12 subjects (66.67%)) and FST IV (3 subjects (16.67%)).

Study visits occurred at baseline, immediately after the first DIAMOND GLOW® treatment with the composition of Example 4, three days after the first DIAMOND GLOW® treatment with the composition of Example 4 and weeks 2, 4, 8, 10 after the DIAMOND GLOW® treatment with the composition of Example 4 and week 12. The following assessments were conducted and results obtained during the study:

Investigator-assessed clinical grading of overall hyperpigmentation, radiance and tactile roughness using a modified Griffiths scale as none (score of 0, best possible condition), mild (1 to 3), moderate (4 to 6), and severe (7 to 9, worst possible condition) were determined.

Melasma severity was also investigator-assessed using the modified Melasma Area and Severity Index (mMASI).

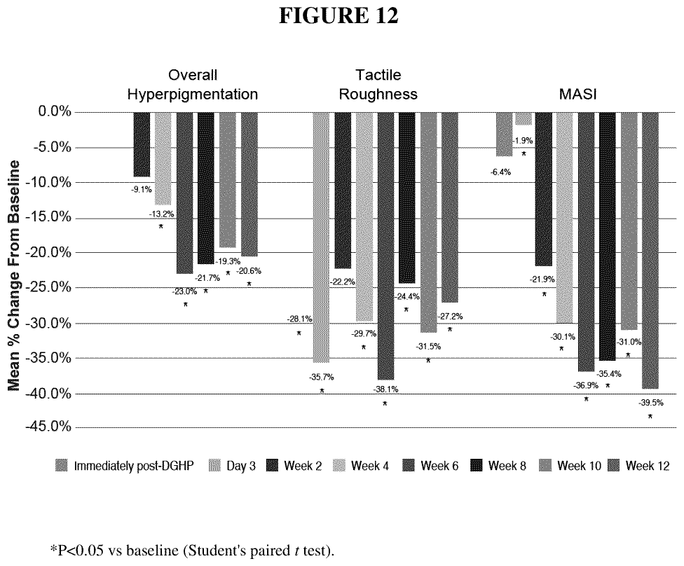

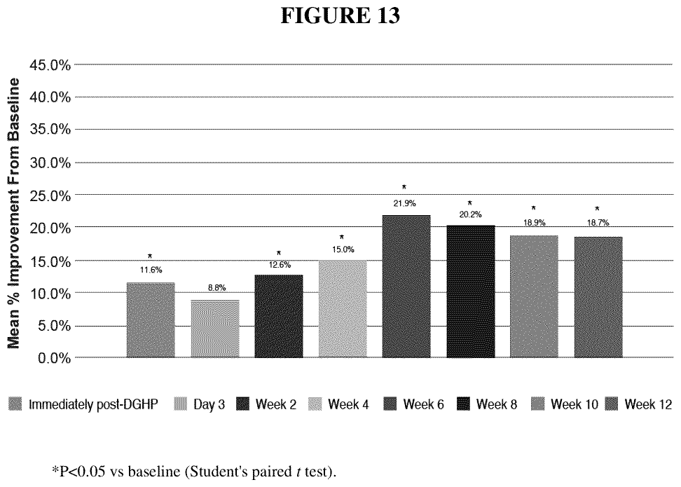

The results of the overall hyperpigmentation, tactile roughness and mMASI assessments are shown in FIG. 12. The results of the radiance assessment is shown in FIG. 13. The results are also shown in the following tables:

Change Versus Baseline in Overall Hyperpigmentation,
MASI, Radiance, and Tactile Roughness Over Time

| | Immediately | | | |
|---|---|---|---|---|
| Investigator | After DG Plus | | | |
| Assessments | EC-DG | Day 3 | Week 2 | Week 4 |
| Overall | NA | NA | −9.1 | ✓ |
| hyperpigmentation | | | | (−13.2%) |
| MASI | −6.4% | ✓ | ✓ | ✓ |
| | | (−1.9%) | (−21.9%) | (−30.1%) |
| Radiance | ✓ | 8.8% | ✓ | ✓ |
| | (11.6%) | | (12.6%) | (15%) |
| Tactile roughness | ✓ | ✓ | −22.2% | ✓ |
| | (−28.1%) | (−35.7%) | | (−29.7%) |

| Investigator | | | | |
|---|---|---|---|---|
| Assessments | Week 6 | Week 8 | Week 10 | Week 12 |
| Overall | ✓ | ✓ | ✓ | ✓ |
| hyperpigmentation | (−23%) | (−21.7%) | (−19.3%) | (−20.6%) |
| MASI | ✓ | ✓ | ✓ | ✓ |
| | (−36.9%) | (−35.4%) | (−31%) | (−39.5%) |
| Radiance | ✓ | ✓ | ✓ | ✓ |
| | (21.9%) | (20.2%) | (18.9%) | (18.7%) |
| Tactile roughness | ✓ | ✓ | ✓ | ✓ |
| | (−38.1%) | (−24.4%) | (−31.5%) | (−27.1%) |

✓ indicates statistically significant improvement vs baseline (p > 0.05)

The above data shows immediately after the first DIAMOND GLOW® treatment with the composition of Example 4, significant improvements versus baseline were observed in radiance and tactile roughness, with continued improvements in these attributes observed as early as day 3 (roughness; p<0.02) and week 2 (radiance; p<0.04) through week 12. Significant improvements from baseline in skin tone evenness were reported at weeks 4 and 6 (mean change of 9.5% and 15.2%, respectively; p<0.04)>

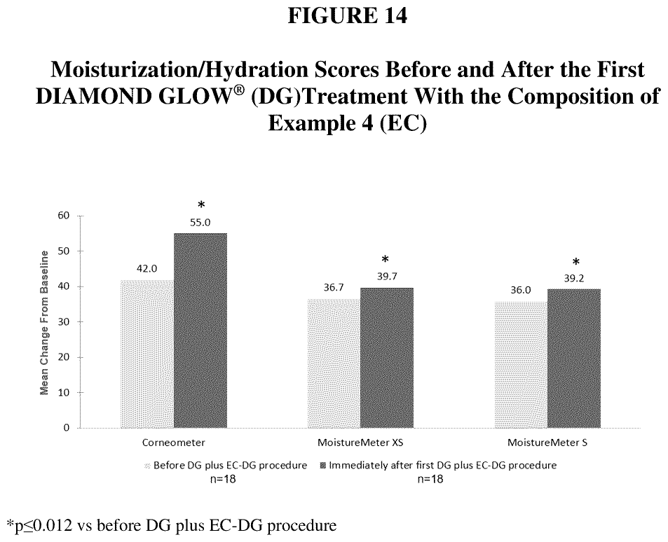

Significant improvements versus baseline immediately after one DIAMOND GLOW® treatment with the composition of Example 4 were observed for moisturization/skin hydration measured by CORNEOMETER (mean change, 13.0) MOISTUREMETER D with XS probe (mean change, 3.0) and MOISTUREMETER D with S probe (mean change, 3.2). All P≤0.012 vs baseline, student's paired t test. The results of the moisturization/skin hydration assessments are shown in FIG. 14.

Antera 3D imaging analysis revealed significant improvements in mean percent change from baseline in brightness of the most-bothersome spot among all subjects at weeks 2, 4, and 6 (all p<0.0095 vs baseline). A significant improvement in pigmentation uniformity (mean change 4.9%), but not pigmentation score or average score was observed from baseline to week 12. Skin roughness score (Ra) decreased significantly from baseline to weeks 4 and 6 (−10.2% and −8.5%, respectively; both p<0.02), indicating an increase in skin smoothness.

Participants completed self-assessment questionnaires immediately following the DIAMOND GLOW® treatment with the composition of Example 4 and at weeks 2, 4, 8, and 12. 93% of the participants were satisfied with the treatment regimen after 2 weeks and onward. A summary of the results of the self-assessment questionnaires immediately following the DIAMOND GLOW® treatment with the composition of Example 4 is provided below:

| | % of Participants Agreed |
|---|---|
| | or Strongly Agreed |
| Reduced the appearance of pores, blackheads, | 94% |
| post-acne scars/marks | |
| Felt my skin received a deep clean/felt my | 94% |
| skin was thoroughly refreshed | |
| Made my skin look refreshed | 89% |
| Made my skin look radiant | 83% |
| Made my skin feel smooth and soft | 94% |
| Made my skin feel hydrated | 89% |

A summary of the results of the self-assessment questionnaires at week 12 is provided below:

| | % of Participants Agreed |
|---|---|
| | or Strongly Agreed |
| Made my skin and pores look clearer | 92% |
| Improved overall appearance of my skin | 100% |
| Gave my skin a healthy glow | 100% |
| Helped me feel more confident about my skin | 100% |
| looks | |
| Made my complexion look more radiant | 100% |
| Made my skin look 5+ years younger | 77% |
| Took 5+ years of sun damage off my skin | 85% |
| Would recommend to a friend | 100% |
| Improved current and helps prevent future | 100% |
| uneven pigment in my skin | |
| Faded the age spots on my skin | 100% |
| Faded even the most stubborn spots on my skin | 75% |
| Reduced the look of sun damage on my skin | 92% |
| Improved the evenness of my skin tone | 100% |
| Faded the dark spots on my skin | 92% |

Tolerability evaluations, including investigator-assessed objective irritation parameters (erythema, edema, and dryness) and participant-reported subjective irritation parameters (burning, stinging, and itching), were performed at each study visit on a 4-point scale, where 0=none, 1=mild, 2=moderate, and 3=severe. Treatment was well tolerated, with mean tolerability scores of ≤1.4 for erythema and <1.0 for dryness, burning/stinging and itching at all time points.

The data provided in this Example demonstrates that the compositions in accordance with the present invention and particularly Example 4 when used in combination with an in-office microdermabrasion treatment provide immediate skin hydration improvements and when used in combination with at-home treatments with compositions of the present invention, particularly Examples 2 and/or 3 are well tolerated, effective in reducing hyperpigmentation and melisma and improving facial roughness and radiance.

The invention described herein may be practiced in the absence of any element or limitation which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1-25. (canceled)

26. A topical composition comprising:
(a) about 0.0005 wt % to about 1.0 wt % of one or more bisbenzylisoquinoline alkaloids;
(b) about 0.05 wt % to about 15 wt % of tranexamic acid;
(c) at least one antioxidant; and
(d) one or more skin conditioning agents.

27. The topical composition of claim 26 wherein the one or more bisbenzylisoquinoline alkaloids are selected from the group consisting of neferine, liensinine, isoliensine, nelumboferine, nelumborine, N-norisoliensinine, 6-hydroxynorisoliensinine and combinations thereof.

28. The topical composition of claim 26 wherein the one or more bisbenzylisoquinoline alkaloids comprises about 0.0005 wt % to about 1.0 wt % of neferine; about 0.0005 wt % to about 1.0 wt %, of liensinine, about 0.0005 wt % to about 1.0 wt %, of isoliensine, or a combination of thereof.

29. The topical composition of claim 28 wherein the composition comprises both neferine and liensinine.

30. The topical composition of claim 26 further comprising one or more additional agents that inhibit the production of melanin selected from the group consisting of resorcinol, phenylethyl resorcinol, tripeptides, tetrapeptide, kojic diplamitate, vitamin C, tetrahexydecyl ascorbate, plankton extracts, sclareolide and combinations thereof.

31. The topical composition of claim 26 further comprising one or more additional agents that inhibit the production of melanin selected from the group consisting of resorcinol, phenylethyl resorcinol or a combination thereof.

32. The topical composition of claim 26 wherein the at least one antioxidant comprises niacinamide.

33. The topical composition of claim 26 further comprising at least one anti-inflammatory agent.

34. The topical composition of claim 26 further comprising at least one exfoliant.

35. The topical composition of claim 26 comprising:
water;
tranexamic acid;
niacinamide;
phytic acid;
phenylethyl resorcinol; and
Vitis vinifera (grape) flower cell extract.

36. The topical composition of claim 35 wherein the one or more bisbenzylisoquinoline alkaloids are present as part of a Nelumbo nucifera germ extract having a neferine concentration of about 0.1% to about 0.2% based on the total weight of the Nelumbo nucifera germ extract and a liensinine concentration of about 0.1% to about 0.2% based on the total weight of the Nelumbo nucifera germ extract.

37. The topical composition of claim 36 comprising:
water;
tranexamic acid;
Nelumbo nucifera germ extract;
niacinamide;
phytic acid;
phenylethyl resorcinol;
Vitis vinifera (grape) flower cell extract;
sclareolide;
sodium hyaluronate;
squalane;
hydroxyacetophenone;
diethyl hexyl syringyldenemalonate; and
xanthophyll.

38. The topical composition of claim 26 comprising:
cyclopentasiloxane;
dimethicone crosspolymer,
water;
niacinamide;
tranexamic acid;
glycolic acid;
tetrahexyldecyl ascorbate;
kojic dipalmitate;
phenylethyl resorcinol; and
acetyl tetrapeptide-2.

39. The topical composition of claim 38 wherein the one or more bisbenzylisoquinoline alkaloids are present as part of a Nelumbo nucifera germ extract having a neferine concentration of about 0.1% to about 0.2% based on the total weight of the Nelumbo nucifera germ extract and a liensinine concentration of about 0.1% to about 0.2% based on the total weight of the Nelumbo nucifera germ extract.

40. The topical composition of claim 39 comprising:
cyclopentasiloxane;
dimethicone crosspolymer,
water;
caprylic/capric triglyceride;
niacinamide;
tranexamic acid;
glycolic acid;
tetrahexyldecyl ascorbate;
kojic dipalmitate;
phenylethyl resorcinol;
diethylhexyl syringylidenemalonate;
bisabolol;
tocopheryl acetate;
allantoin;
Nelumbo nucifera germ extract;
dipotassium glycyrrhizate;
squalane;
ceramide; and
acetyl tetrapeptide-2.

41. The topical composition of claim 26 comprising:
water;
tranexamic acid;
glycolic acid;
phytic acid;
Citrullus lanatus (watermelon) fruit extract;
lens esculenta (lentil) fruit extract;
pyrus malus (apple) fruit extract;
niacinamide;
Laminaria digitata extract;
Artemisia vulgaris extract; and
Chamomilla recutita (matricaria) flower extract.

42. The topical composition of claim 41 wherein the one or more bisbenzylisoquinoline alkaloids are present as part of a Nelumbo nucifera germ extract having a neferine concentration of about 0.1% to about 0.2% based on the total weight of the Nelumbo nucifera germ extract and a liensinine concentration of about 0.1% to about 0.2% based on the total weight of the nelumbo nucifera germ extract.

43. The topical composition of claim 42 comprising:
water;
aloe barbadensis leaf extract;
tranexamic acid;
glycolic acid;
phytic acid;
Citrullus lanatus (watermelon) fruit extract;
panthenol;
lens esculenta (lentil) fruit extract;
pyrus malus (apple) fruit extract;
niacinamide;
allantoin;
sodium hyaluronate;
sodium PCA;
Nelumbo nucifera germ extract;
Laminaria digitata extract;
Artemisia vulgaris extract; and
Chamomilla recutita (matricaria) flower extract.

44. The topical composition of claim 26 comprising:
water;
Hamamelis virginiana (witch hazel) water;
betaine;
niacinamide;
tranexamic acid; and
glycolic acid.

45. The topical composition of claim 44 wherein the one or more bisbenzylisoquinoline alkaloids are present as part of a Nelumbo nucifera germ extract having a neferine concentration of about 0.1% to about 0.2% based on the total weight of the Nelumbo nucifera germ extract and a liensinine concentration of about 0.1% to about 0.2% based on the total weight of the Nelumbo nucifera germ extract.

46. The topical composition of claim 45 comprising:
water;
Hamamelis virginiana (witch hazel) water,
betaine;
glycolic acid;
niacinamide;
tranexamic acid;
dipotassium glycyrrhizinate; and
Nelumbo nucifera germ extract.

47. A method for treating melanin or pigmentation disorders comprising the applying the topical composition of claim 26 to the outer skin surface of a human in need of such treatment.

48. The method of claim 47 wherein the topical composition is applied in combination with an in-office procedure selected from the groups consisting of chemical peel, laser/light therapy, micro needling and/or microdermabrasion.

49. The method of claim 47 wherein the topical composition is applied at least once a day for 2 weeks.

50. The method of claim 47 wherein the topical composition is applied at least twice a day for 2 weeks.
